(19) United States
(12) Patent Application Publication
(10) Pub. No.: US 2025/0049765 A1
(43) Pub. Date: Feb. 13, 2025

(54) BICYCLO [3.2.0] HEPTANE BIS(AMIDE) RXFP1 AGONISTS

(71) Applicant: BRISTOL-MYERS SQUIBB COMPANY, Princeton, NJ (US)

(72) Inventors: Andrew K. Dilger, Golden, CO (US); Leon M. Smith ll, Somerset, NJ (US); Michael J. Orwat, New Hope, PA (US); Donald J.P. Pinto, Churchville, PA (US)

(21) Appl. No.: 18/718,484

(22) PCT Filed: Dec. 14, 2022
(86) PCT No.: PCT/US2022/081522

§ 371 Date: Jun. 11, 2024

Related U.S. Application Data
(60) Provisional application No. 63/289,859, filed on Dec. 15, 2021.

(51) Int. Cl. A61K 31/423; A61K 31/27; C07C 237/24; C07D 261/04; C07D 261/20

(52) U.S. Cl. CPC A61K 31/423; A61K 31/27; C07C 237/24; C07D 261/04; C07D 261/20

(57) ABSTRACT

The disclosure relates to compounds of Formula (I), which are RXFP1 receptor agonists, compositions containing them, and methods of using them, for example, in the treatment of heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension).

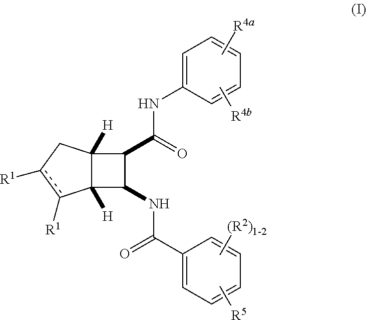

### CROSS-REFERENCE TO RELATED APPLICATIONS

This application claims the benefit of U.S. Provisional Application No. 63/289,859, filed Dec. 15, 2021, the disclosure of which is incorporated herein by reference in its entirety.

### BACKGROUND OF THE INVENTION

The present disclosure relates to novel compounds which are relaxin family peptide receptor 1 (RXFP1) agonists, compositions containing them, and methods of using them, for example in the treatment of heart failure, fibrotic diseases, and related diseases such as lung disease (e.g., idiopathic pulmonary fibrosis), kidney disease (e.g., chronic kidney disease), and hepatic disease (e.g., non-alcoholic steatohepatitis and portal hypertension).

The human relaxin hormone (also called relaxin or H2 relaxin) is a 6-kDa peptide composed of 53 amino acids whose activity was initially discovered when Frederick Hisaw in 1926 injected crude extracts from swine corpus luteum into virgin guinea pigs and observed a relaxation of the fibrocartilaginous pubic symphysis joint (Hisaw F L., Proc. Soc. Exp. Biol. Med., 1926, 23, 661-663). The relaxin receptor was previously known as Lgr7 but is now officially termed the relaxin family peptide receptor 1 (RXFP1) and was deorphanized as a receptor for relaxin in 2002 (Hsu S Y., et al., Science, 2002, 295, 671-674). RXFP1 is reasonably well conserved between mouse and human with 85% amino acid identity and is essentially ubiquitously expressed in humans and in other species (Halls M L., et al., Br. J. Pharmacol., 2007, 150, 677-691). The cell signaling pathways for relaxin and RXFP1 are cell type dependent and quite complex (Halls M L., et al., Br. J. Pharmacol., 2007, 150, 677-691; Halls M L., et al. Ann. N Y Acad. Sci., 2009, 1160, 108-111; Halls M L., Ann N Y Acad. Sci., 2007, 1160, 117-120). The best studied pathway is the relaxin-dependent increase in cellular levels of CAMP in which relaxin functions as an RXFP1 agonist to promote GαS coupling and activation of adenylate cyclase (Halls M L., et al., Mol. Pharmacol., 2006, 70, 214-226).

Since the initial discovery of relaxin much experimental work has focused on delineating the role relaxin has played in female reproductive biology and the physiological changes that occur during mammalian pregnancy (Sherwood O D., Endocr. Rev., 2004, 25, 205-234). During human gestation, in order to meet the nutritional demands imposed upon it by the fetus, the female body undergoes a significant ˜30% decrease in systemic vascular resistance (SVR) and a concomitant ˜50% increase in cardiac output (Jeyabalan A C., K. P., Reanl and Electolyte Disorders. 2010, 462-518), (Clapp J F. & Capeless E., Am. J. Cardio., 1997, 80, 1469-1473). Additional vascular adaptations include an ˜30% increase in global arterial compliance that is important for maintaining efficient ventricular-arterial coupling, as well as an ˜50% increase in both renal blood flow (RBF) and glomerular filtration rate (GFR), important for metabolic waste elimination (Jeyabalan A C., K. P., Reanl and Electolyte Disorders. 2010, 462-518). (Poppas A., et al., Circ., 1997, 95, 2407-2415). Both pre-clinical studies in rodents as well as clinical studies performed in a variety of patient settings, provide evidence that relaxin is involved, at least to some extent, in mediating these adaptive physiological changes (Conrad K P., Regul. Integr. Comp. Physiol., 2011, 307. R267-275), (Teichman S L., et al., Heart Fail. Rev., 2009, 14, 321-329). Importantly, many of these adaptive responses would likely be of benefit to HF patients in that excessive fibrosis, poor arterial compliance, and poor renal function are all characteristics common to heart failure patients (Mohammed S F., et al., Circ., 2015, 131, 550-559). (Wohlfahrt P., et al., Eur. J. Heart Fail., 2015, 17, 27-34), (Damman K., et al., Prog. Cardiovasc. Dis., 2011, 54, 144-153).

Heart failure (HF), defined hemodynamically as “systemic perfusion inadequate to meet the body's metabolic demands as a result of impaired cardiac pump function”, represents a tremendous burden on today's health care system with an estimated United States prevalence of 5.8 million and greater than 23 million worldwide (Roger V L., et al., Circ. Res., 2013, 113, 646-659). It is estimated that by 2030, an additional 3 million people in the United States alone will have HF, a 25% increase from 2010. The estimated direct costs (2008 dollars) associated with HF for 2010 was $25 billion, projected to grow to $78 B by 2030 (Heidenreich P A., et al., Circ., 2011, 123, 933-944). Astoundingly, in the United States, I in 9 deaths has HF mentioned on the death certificate (Roger V L., et al., Circ., 2012, 125, e2-220) and, while survival after HF diagnosis has improved over time (Matsushita K., et al., Diabetes, 2010, 59, 2020-2026), (Roger V L., et al., JAMA, 2004, 292, 344-350), the death rate remains high with ˜50% of people with HF dying within 5 years of diagnosis (Roger V L., et al., Circ., 2012, 125, e2-220), (Roger V L., et al., JAMA, 2004, 292, 344-350).

The symptoms of HF are the result of inadequate cardiac output and can be quite debilitating depending upon the advanced stage of the disease. Major symptoms and signs of HF include: 1) dyspnea (difficulty in breathing) resulting from pulmonary edema due to ineffective forward flow from the left ventricle and increased pressure in the pulmonary capillary bed; 2) lower extremity edema occurs when the right ventricle is unable to accommodate systemic venous return; and 3) fatigue due to the failing heart's inability to sustain sufficient cardiac output (CO) to meet the body's metabolic needs (Kemp C D., & Conte J V., Cardiovasc. Pathol., 2011, 21, 365-371). Also, related to the severity of symptoms, HF patients are often described as “compensated” or “decompensated”. In compensated heart failure, symptoms are stable, and many overt features of fluid retention and pulmonary edema are absent. Decompensated heart failure refers to a deterioration, which may present as an acute episode of pulmonary edema, a reduction in exercise tolerance, and increasing breathlessness upon exertion (Millane T., et al., BMJ, 2000, 320, 559-562).

In contrast to the simplistic definition of poor cardiac performance not being able to meet metabolic demands, the large number of contributory diseases, multitude of risk factors, and the many pathological changes that ultimately lead to heart failure make this disease exceedingly complex (Jessup M. & Brozena S., N. Engl. J. Med., 2003, 348, 3007-2018). Injurious events thought to be involved in the pathophysiology of HF range from the very acute such as myocardial infarction to a more chronic insult such as life-long hypertension. Historically, HF was primarily described as “systolic HF” in which decreased left-ventricular (LV) contractile function limits the expulsion of blood and hence results in a reduced ejection fraction (EF is stroke volume/end diastolic volume), or “diastolic HF” in which active relaxation is decreased and passive stiffness is increased limiting LV filling during diastole, however overall EF is maintained (Borlaug B A. & Paulus W J., Eur Heart J., 2011, 32, 670-679). More recently, as it became understood that diastolic and systolic LV dysfunction was not uniquely specific to these two groups, new terminology was employed: “heart failure with reduced ejection fraction” (HFrEF), and “heart failure with preserved ejection fraction” (HFpEF) (Borlaug B A. & Paulus W J., Eur Heart J., 2011, 32, 670-679). Although these two patient populations have very similar signs and symptoms, whether HFrEF and HFpEF represent two distinct forms of HF or two extremes of a single spectrum sharing a common pathogenesis is currently under debate within the cardiovascular community (Borlaug B A. & Redfield M M., Circ., 2011, 123, 2006-2013), (De Keulenaer G W., & Brutsaert D L., Circ., 2011, 123, 1996-2004).

Serelaxin, an intravenous (IV) formulation of the recombinant human relaxin peptide with a relatively short first-phase pharmacokinetic half-life of 0.09 hours, is currently being developed for the treatment of HF (Novartis, 2014). Serelaxin has been given to normal healthy volunteers (NHV) and demonstrated to increase RBF (Smith M C., et al., J. Am. Soc. Nephrol. 2006, 17, 3192-3197) and estimated GFR (Dahlke M., et al., J. Clin. Pharmacol., 2015, 55, 415-422). Increases in RBF were also observed in stable compensated HF patients (Voors A A., et al., Cir. Heart Fail., 2014, 7, 994-1002). In large clinical studies, favorable changes in worsening renal function, worsening HF, as well as fewer deaths, were observed in acute decompensated HF (ADHF) patients in response to an in-hospital 48 hour IV infusion of serelaxin (Teerlink J R., et al., Lancet, 2013, 381, 29-39), (Ponikowski P., et al., Eur. Heart. 2014, 35, 431-441). Suggesting that chronic dosing of serelaxin could provide sustained benefit to HF patients, improvement in renal function based on serum creatinine levels was observed in scleroderma patients given serelaxin continuously for 6 months using a subcutaneous pump (Teichman S L., et al., Heart Fail. Rev., 2009, 14, 321-329). In addition to its potential as a therapeutic agent for the treatment of HF, continuous subcutaneous administration of relaxin has also been demonstrated to be efficacious in a variety of animal models of lung (Unemori E N., et al., J. Clin. Invet., 1996, 98, 2739-2745), kidney (Garber S L., et al., Kidney Int., 2001, 59, 876-882), and liver injury (Bennett R G., Liver Int., 2014, 34, 416-426).

In summary, a large body of evidence supports a role for relaxin-dependent agonism of RXFP1 mediating the adaptive changes that occur during mammalian pregnancy, and that these changes translate into favorable physiological effects and outcomes when relaxin is given to HF patients. Additional preclinical animal studies in various disease models of lung, kidney, and liver injury provide evidence that relaxin, when chronically administered, has the potential to provide therapeutic benefit for multiple indications in addition to HF. More specifically, chronic relaxin administration could be of benefit to patients suffering from lung disease (e.g., idiopathic pulmonary fibrosis), kidney disease (e.g., chronic kidney disease), or hepatic disease (e.g., non-alcoholic steatohepatitis and portal hypertension).

### SUMMARY OF THE INVENTION

The present invention provides novel substituted cyclobutylcyclopentane compounds, their analogues, including stereoisomers, tautomers, pharmaceutically acceptable salts, or solvates thereof, which are useful as RXFP1 receptor agonists.

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, pharmaceutically acceptable salts, or solvates thereof.

The compounds of the invention may be used, for example, in the treatment and/or prophylaxis of heart failure, fibrotic diseases, and related diseases, such as; lung disease (e.g., idiopathic pulmonary fibrosis), kidney disease (e.g., chronic kidney disease), or hepatic disease (e.g., non-alcoholic steatohepatitis and portal hypertension).

The compounds of the present invention may be used in therapy.

The compounds of the present invention may be used for the manufacture of a medicament for the treatment and/or prophylaxis of heart failure.

The compounds of the invention can be used alone, in combination with other compounds of the present invention, or in combination with one or more, preferably one to two other agent(s).

These and other features of the invention will be set forth in expanded form as the disclosure continues.

### DESCRIPTION OF THE INVENTION

The invention encompasses compounds of Formula (I), which are RXFP1 receptor agonists, compositions containing them, and methods of using them.

In a first aspect, the present invention provides, inter alia, compounds of Formula (1):

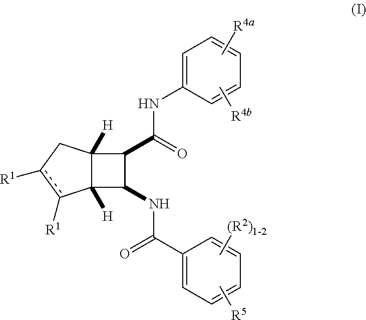

or pharmaceutically acceptable salts thereof, wherein:
[symbol] is an optional bond;
R¹ is H or halo; or R¹ and R¹ together form a phenyl ring;
R² is halo, C₁₋₄ alky, OH, or —OC₁₋₄ alkyl substituted with 0-4 halo, OH, or —OC₁₋₄ alkyl;
R^{4a} is halo;
R^{4b} is C₁₋₄ alkyl substituted with 0-4 halo;
R⁵ is C₂₋₈ alkenyl substituted with 0-3 R⁶ and 0-2 R⁷, C₂₋₈ alkynyl substituted with 0-3 R⁶ and 0-2 R⁷, C₆₋₁₂ aryl substituted with 0-3 R⁶ and 0-2 R², or a 3- to 12-membered heterocyclyl comprising 1-4 heteroatoms selected from O, S(═O)ₚ, N and NR¹⁰ substituted with 0-3 R⁶ and 0-1 R⁷; wherein said heterocyclyl is bonded to the phenyl moiety through a carbon or nitrogen atom;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{a}, —OC(═O)R^{a}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR⁴, and substituted with 0-5 R^{e};
R⁸ is halo, —C(═O)OR^{b}, —C(═O)NHR^{a}, —C(═O)NHOR^{b}, or C₁-alkyl substituted with 0-3 halo or OH;
R⁹ is —OR^{b}, —NR^{a}R^{a}, —NR^{a}C(═O)R^{b}, —NR^{a}C(═O)OR⁶, —NR^{a}S(═O)ₚR^{e}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₆ alkyl substituted with 0-5 R^{e}, C₂₋₅ alkenyl substituted with 0-5 R^{e}, C₂₋₆ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃alkyl;
R^{g} is halo, CN, OH, C₁₋₆ alkyl, C₃₋₆ cycloalkyl, or aryl;
n is zero, 1, 2, or 3; and
p is zero, 1, or 2.

In a second aspect within the scope of the first aspect, the present invention provides compounds of Formula (II):

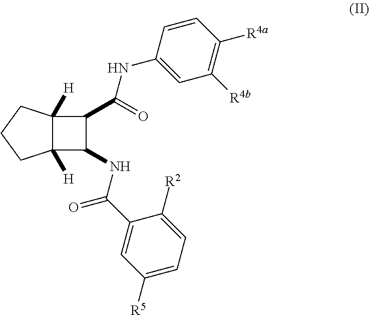

or pharmaceutically acceptable salts thereof, wherein:
R² is —OC₁₋₄ alkyl substituted with 0-4 halo;
R^{4a} is halo;
R^{4b} is C₁₋₃ alkyl substituted with 0-4 F;
R⁵ is C₂₋₆ alkynyl substituted with 0-3 R⁶ and 0-2 R⁷, C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, CN, C is alkyl, —OH, or —OC₁₋₄ alkyl;
R⁷ is C₁₋₂ alkyl substituted with 0-1 R⁸ and 0-1 R⁹, OR^{b}, —NR^{a}R^{a}, —NR^{a}C(═O)R^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR⁴, and substituted with 0-4 R^{e};
R⁸ is halo, —C(═O)OR⁶, —C(═O)NHR^{a}, —C(═O)NHOR^{b}, or C₁₋₄ alkyl substituted with 0-3 halo or OH;
R⁹ is-OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR³OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, or —C(═O)NR^{a}R^{a};
R^{a} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₂₋₅ alkenyl substituted with 0-4 R^{e}, C₂₋₅ alkynyl substituted with 0-4 R^{e}, C₃₋₆ carbocyclyl, or heterocyclyl;
R^{d} is H or C₁₋₂ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl;
R^{g} is halo, CN, OH, C₁₋₆ alkyl, or C₃₄ cycloalkyl;
n is zero, 1, 2, or 3; and
p is zero, 1, or 2.

In a third aspect within the scope of the first and second aspects, the present invention provides compounds of Formula (III):

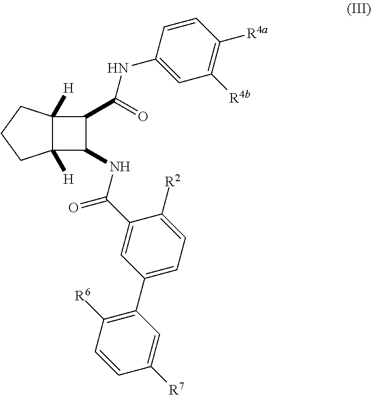

or pharmaceutically acceptable salts thereof, wherein:
R² is —OC₁₋₃ alkyl;
R^{4a} is F;
R^{4b} is CF₃;
R⁶ is halo;
R⁷ is C₁₋₂ alkyl substituted with 0-1 R⁸ and 0-1 R⁹, —C(═O)OR^{b}, or —C(═O)NR^{a}R^{a};
R⁸ is —C(═O)OR^{b}, —C(═O)NHR^{a}, or C₁₋₄ alkyl substituted with 0-3 halo or OH;
R⁹ is —OR^{b}, —NR^{a}R^{a}, —NR^{a}C(═O)R^{b}, or —OC(═O)NR^{a}R^{a};
R^{a} is H, C₁₋₄ alkyl substituted with 0-3 R^{e}, —(CH₂)ₙ—C₃₋₆ cycloalkyl substituted with 0-3 R^{e}, or phenyl substituted with 0-3 R^{e};
R^{b} is H or heterocyclyl substituted with 0-3 R^{e};
R^{c} is halo, CN, ═O, or C₁₋₆ alkyl; and
n is zero or 1.

In a fourth aspect within the scope of the second aspect, the present invention provides compounds of Formula (II) or pharmaceutically acceptable salts thereof, wherein:
R² is —OCH₃;
R^{4a} is F;
R^{4b} is CF₃;
R⁵ is

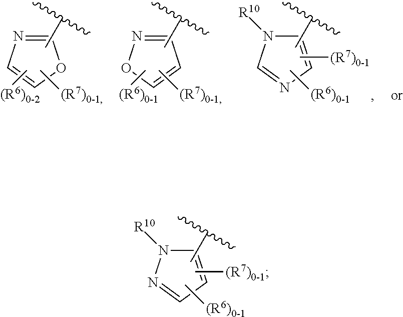

R⁶ is halo, —OH, or C₁₋₄ alkyl substituted with 0-1 OH;
R⁷ is C₁₋₂ alkyl substituted with 0-1 R⁸ and 0-1 R⁹;
R⁸ is —C(═O)OR^{b}, —C(═O)NHR^{a}, or —C(═O)NHOR^{b};
R⁹ is —OR^{b} or —NR^{a}R^{a};
R¹⁰ is H, —C(═O)R^{b}, or C₁₋₄ alkyl substituted with 0-1R¹¹;
R¹¹ is —OH, —C(═O)OH, or aryl;
R^{a} is H or C₁₋₃ alkyl; and
R^{b} is H or C₁₋₃ alkyl.

In a fifth aspect within the scope of the second aspect, the present invention provides compounds of Formula (II) or pharmaceutically acceptable salts thereof, wherein:
R² is —OCH₃;
R^{4a} is F;
R^{4b} is CF₃;
R⁵ is

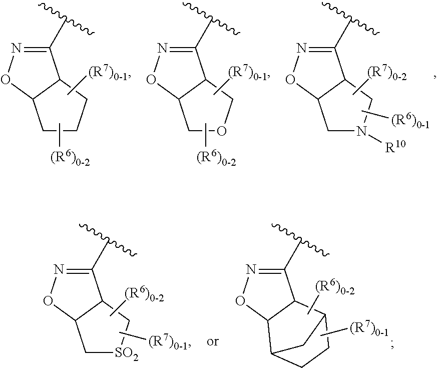

R⁶ is halo, C₁₋₄ alkyl, —OH, or —OC₁₋₄ alkyl;
R⁷ is C₁₋₄ alkyl substituted with 0-1 R⁸ and 0-1 R⁹;
R⁸ is —C(═O)OR′;
R⁹ is OH;
R¹⁰ is H, C₁₋₃ alkyl substituted with 0-2 R¹¹, or —C(═O) OC₁₋₄ alkyl;
R¹¹ is —OH, —C(═O)OH, or aryl; and
R^{b} is H or C₁₋₃ alkyl.

In a sixth aspect within the scope of the second aspect, the present invention provides compounds of Formula (II), or pharmaceutically acceptable salts thereof, wherein:
R² is —OCH₃;
R^{4a} is F;
R⁴ is CF₃;
R⁵ is C₂₋₅ alkynyl substituted with 0-1 R⁷;
R⁷ is —OR^{b};
R^{b} is H, C₁₋₃ alkyl, or phenyl substituted with 0-2 R^{e};
R^{e} is halo, C₁₋₃ alkyl, or C(═O)OR^{f}; and
R^{f} is H or C₁₋₃ alkyl.

For a compound of Formula (I), the scope of any instance of a variable substituent, including R¹, R², R³, R^{4a}, R^{4b}, R⁵, R⁶, R⁷, R⁸, R⁹, R¹⁰, R¹¹, R¹², R^{a}, R^{b}, R^{c}, R^{d}, R^{e}, R^{f}, and R^{g} can be used independently with the scope of any other instance of a variable substituent. As such, the invention includes combinations of the different aspects.

In one embodiment of Formula (II) or (III), R^{4a} is F.

In another embodiment of Formula (II) or (III), R^{4b} is CF₃.

In one embodiment of Formula (III), R² is —OCH₃; R^{4a} is F; R^{4b} is CF₃; R⁵ is

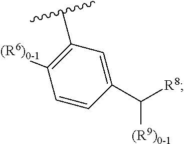

R⁶ is F; R⁸ is —C(═O)OH, —C(═O)NHR^{a}, or CF₃; R⁹ is —NHR^{a}, —NHC(═O)R^{b}, —NHS(═O)ₚC₁₋₄ alkyl or —OC(═O)NHR^{a}; R^{a} is H, C₁₋₃ alkyl, —(CH₂)₀₋₁—C₃₋₆ cycloalkyl, or —(CH₂)₀₋₁-phenyl substituted with 0-2 R^{e}; R^{b} is H or heterocyclyl; R^{e} is C₁₋₃ alkyl, —(CH₂)₀₋₁OR^{f}; and R^{f} is H or C₁₋₃ alkyl.

In one embodiment of Formula (II). R² is —OCH₃; R^{4a} is F; R^{4b} is CF₃; R⁵ is

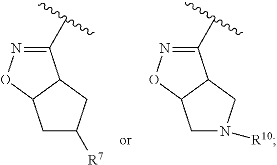

R⁷ is C₁₋₄ alkyl substituted with 0-1 R⁹; R⁹ is —OH; R¹⁰ is —C(═O)R⁶; R^{b} is H or C₁₋₃ alkyl substituted with 0-4 R^{e}; R^{e} is —(CH₂)₀₋₁OR^{f}; and R^{f} is H or C₁₋₃ alkyl.

Unless specified otherwise, these terms have the following meanings.

“Halo” includes fluoro, chloro, bromo, and iodo.

“Alkyl” or “alkylene” is intended to include both branched and straight-chain saturated aliphatic hydrocarbon groups having the specified number of carbon atoms. For example, “C₁ to C₁₀ alkyl” or “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁ to C₆ alkyl” or “C₁-C₆ alkyl” denotes alkyl having 1 to 6 carbon atoms. Alkyl group can be unsubstituted or substituted with at least one hydrogen being replaced by another chemical group. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl). When “C₀ alkyl” or “C₀ alkylene” is used, it is intended to denote a direct bond. “Alkyl” also includes deuteroalkyl such as CD₃.

“Alkenyl” or “alkenylene” is intended to include hydrocarbon chains of either straight or branched configuration having one or more, preferably one to three, carbon-carbon double bonds that may occur in any stable point along the chain. For example, “C₂ to C₆ alkenyl” or “C₂₋₆ alkenyl” (or alkenylene), is intended to include C₂, C₃, C₄, C₅, and C₆ alkenyl groups; such as ethenyl, propenyl, butenyl, pentenyl, and hexenyl.

“Alkynyl” or “alkynylene” is intended to include hydrocarbon chains of either straight or branched configuration having one or more, preferably one to three, carbon-carbon triple bonds that may occur in any stable point along the chain. For example, “C₂ to C₆ alkynyl” or “C₂₋₆ alkynyl” (or alkynylene), is intended to include C₂, C₃, C₄, C₅, and C₆ alkynyl groups; such as ethynyl, propynyl, butynyl, pentynyl, and hexynyl.

“Carbocycle”, “carbocyclyl”, or “carbocyclic residue” is intended to mean any stable 3-, 4-, 5-, 6-, 7-, or 8-membered monocyclic or bicyclic or 7-, 8-, 9-, 10-, 11-, 12-, or 13-membered bicyclic or tricyclic hydrocarbon ring, any of which may be saturated, partially unsaturated, unsaturated or aromatic. Examples of such carbocyclyls include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocyclyl (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. When the term “carbocyclyl” is used, it is intended to include “aryl,” “cycloalkyl,” and “spirocycloalkyl.” Preferred carbocyclyls, unless otherwise specified, are cyclopropyl, cyclobutyl, cyclopentyl, cyclohexyl, phenyl, and indanyl.

“Cycloalkyl” is intended to mean cyclized alkyl groups, including mono-, bi- or multicyclic ring systems. “C₃ to C₇ cycloalkyl” or “C₃₋₇ cycloalkyl” is intended to include C₃, C₄, C₅, C₆, and C₇ cycloalkyl groups. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl.

“Spirocycloalkyl” is intended to mean hydrocarbon bicyclic ring systems with both rings connected through a single atom. The ring can be different in size and nature, or identical in size and nature. Examples include spiropentane, spriohexane, spiroheptane, spirooctane, spirononane, or spirodecane.

“Bicyclic carbocyclyl” or “bicyclic carbocyclic group” is intended to mean a stable 9- or 10-membered carbocyclic ring system that contains two fused rings and consists of carbon atoms. Of the two fused rings, one ring is a benzo ring fused to a second ring; and the second ring is a 5- or 6-membered carbon ring which is saturated, partially unsaturated, or unsaturated. The bicyclic carbocyclic group may be attached to its pendant group at any carbon atom which results in a stable structure. The bicyclic carbocyclic group described herein may be substituted on any carbon if the resulting compound is stable. Examples of a bicyclic carbocyclic group are, but not limited to, naphthyl, 1,2-dihydronaphthyl, 1,2,3,4-tetrahydronaphthyl, and indanyl.

“Aryl” groups refer to monocyclic or polycyclic aromatic hydrocarbons, including, for example, phenyl, naphthyl, and phenanthranyl. Aryl moieties are well known and described, for example, in Lewis. R. J., ed., Hawley's Condensed Chemical Dictionary, 13th Edition, John Wiley & Sons, Inc., New York (1997).

“Benzyl” is intended to mean a methyl group on which one of the hydrogen atoms is replaced by a phenyl group, wherein said phenyl group may optionally be substituted with 1 to 5 groups, preferably 1 to 3 groups.

“Heterocycle”, “heterocyclyl” or “heterocyclic ring” is intended to mean a stable 3-, 4-, 5-, 6-, or 7-membered monocyclic or bicyclic or 7-, 8-, 9-, 10-, 11-, 12-, 13-, or 14-membered polycyclic heterocyclic ring that is saturated, partially unsaturated, or fully unsaturated, and that contains carbon atoms and 1, 2, 3 or 4 heteroatoms independently selected from the group consisting of N, O and S; and including any polycyclic group in which any of the above-defined heterocyclic rings is fused to a benzene ring. The nitrogen and sulfur heteroatoms may optionally be oxidized (i.e., N→O and S(O)ₚ, wherein p is 0, 1 or 2). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocyclyl may optionally be quaternized. It is preferred that when the total number of S and O atoms in the heterocyclyl exceeds 1, then these heteroatoms are not adjacent to one another. It is preferred that the total number of S and O atoms in the heterocyclyl is not more than 1. Bridged rings are also included in the definition of heterocyclyl. When the term “heterocyclyl” is used, it is intended to include heteroaryl.

Examples of heterocyclyls include, but are not limited to, acridinyl, azetidinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, imidazolopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocyclyls.

“Bicyclic heterocyclyl” “bicyclic heterocyclyl” or “bicyclic heterocyclic group” is intended to mean a stable 9- or 10-membered heterocyclic ring system which contains two fused rings and consists of carbon atoms and 1, 2, 3, or 4 heteroatoms independently selected from the group consisting of N, O and S. Of the two fused rings, one ring is a 5- or 6-membered monocyclic aromatic ring comprising a 5-membered heteroaryl ring, a 6-membered heteroaryl ring or a benzo ring, each fused to a second ring. The second ring is a 5- or 6-membered monocyclic ring which is saturated, partially unsaturated, or unsaturated, and comprises a 5-membered heterocyclyl, a 6-membered heterocyclyl or a carbocyclyl (provided the first ring is not benzo when the second ring is a carbocyclyl).

The bicyclic heterocyclic group may be attached to its pendant group at any heteroatom or carbon atom which results in a stable structure. The bicyclic heterocyclic group described herein may be substituted on carbon or on a nitrogen atom if the resulting compound is stable. It is preferred that when the total number of S and O atoms in the heterocyclyl exceeds 1, then these heteroatoms are not adjacent to one another. It is preferred that the total number of S and O atoms in the heterocyclyl is not more than 1.

Examples of a bicyclic heterocyclic group are, but not limited to, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydroquinolinyl, 2,3-dihydrobenzofuranyl, chromanyl, 1,2,3,4-tetrahydroquinoxalinyl, and 1,2,3,4-tetrahydroquinazolinyl.

“Heteroaryl” is intended to mean stable monocyclic and polycyclic aromatic hydrocarbons that include at least one heteroatom ring member such as sulfur, oxygen, or nitrogen, Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O and S(O)ₚ, wherein p is 0, 1 or 2).

As referred to herein, the term “substituted” means that at least one hydrogen atom is replaced with a non-hydrogen group, provided that normal valencies are maintained and that the substitution results in a stable compound. When a substituent is keto (i.e., =0), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C═C, C═N, or N═N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→0) derivative.

When any variable occurs more than one time in any constituent or formula for a compound, its definition at each occurrence is independent of its definition at every other occurrence. Thus, for example, if a group is shown to be substituted with 0-3 R groups, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The invention includes all tautomeric forms of the compounds, atropisomers and rotational isomers.

All processes used to prepare compounds of the present invention and intermediates made therein are considered to be part of the present invention.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996))

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

The invention is intended to include all isotopes of atoms occurring in the compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans- (or E- and Z—) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

### Biological Methods

RXFP1 Cyclic Adenosine Monophosphate (CAMP) Assays. Human embryonic kidney cells 293 (HEK293) cells and HEK293 cells stably expressing human RXFP1, were cultured in MEM medium supplemented with 10% qualified FBS, and 300 μg/ml hygromycin (Life Technologies). Cells were dissociated and suspended in assay buffer. The assay buffer was HBSS buffer (with calcium and magnesium) containing 20 mM HEPES, 0.05% BSA, and 0.5 mM IBMX. Cells (3000 cells per well, except 1500 cell per well for HEK293 cells stably expressing human RXFP1) were added to 384-well Proxiplates (Perkin-Elmer). Cells were immediately treated with test compounds in DMSO (2% final) at final concentrations in the range of 0.010 nM to 50 μM. Cells were incubated for 30 min at room temperature. The level of intracellular cAMP was determined using the HTRF HiRange CAMP assay reagent kit (Cisbio) according to manufacturer's instructions. Solutions of cryptate conjugated anti-cAMP and d2 fluorophore-labelled cAMP were made in a supplied lysis buffer separately. Upon completion of the reaction, the cells were lysed with equal volume of the d2-cAMP solution and anti-cAMP solution. After a 1 h room temperature incubation, time-resolved fluorescence intensity was measured using the Envision (Perkin-Elmer) at 400 nm excitation and dual emission at 590 nm and 665 nm. A calibration curve was constructed with an external cAMP standard at concentrations ranging from 2.7 μM to 0.1 μM by plotting the fluorescent intensity ratio from 665 nm emission to the intensity from the 590 nm emission against cAMP concentrations. The potency and activity of a compound to inhibit cAMP production was then determined by fitting to a 4-parametric logistic equation from a plot of cAMP level versus compound concentrations.

The examples disclosed below were tested in the human RXFP1 (hRXFP1) HEK293 cAMP assay described above and found to have agonist activity. Table A lists EC₅₀ values in the hRXFP1 HEK293 cAMP assay measured for the examples.

TABLE A

| | cAMP hRXFP1 |
|---|---|
| | HEK293 Assay |
| Example # | EC₅₀ (nM) |
| 1 | 1,193 |
| 2 | 823 |
| 3 | 916 |
| 5 | 2,232 |
| 7 | 12 |

### Pharmaceutical Compositions and Methods of Use

The compounds of Formula (I) are RXFP1 receptor agonists and may find use in the treatment of medical indications such as heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension).

Another aspect of the invention is a pharmaceutical composition comprising a compound of Formula (I) and a pharmaceutically acceptable carrier.

Another aspect of the invention is a pharmaceutical composition comprising a compound of Formula (I) for the treatment of a relaxin-associated disorder and a pharmaceutically acceptable carrier.

Another aspect of the invention is a method of treating a disease associated with relaxin comprising administering an effective amount of a compound of Formula (I).

Another aspect of the invention is a method of treating a cardiovascular disease comprising administering an effective amount of a compound of Formula (I) to a patient in need thereof.

Another aspect of the invention is a method of treating heart failure comprising administering an effective amount of a compound of Formula (I) to a patient in need thereof.

Another aspect of the invention is a method of treating fibrosis comprising administering a therapeutically effective amount of a compound of Formula (I) to a patient in need thereof.

Another aspect of the invention is a method of treating a disease associated with fibrosis comprising administering a therapeutically effective amount of a compound of Formula (1) to a patient in need thereof.

Another aspect of the invention is a method of treating or preventing kidney failure, comprising administering a therapeutically effective amount of a compound of Formula (I) to a patient in need thereof.

Another aspect of the invention is a method of improving, stabilizing or restoring renal function in a patient in need thereof, comprising administering a therapeutically effective amount of a compound of Formula (I) to the patient.

Unless otherwise specified, the following terms have the stated meanings.

The term “patient” or “subject” refers to any human or non-human organism that could potentially benefit from treatment with a RXFP1 agonist as understood by practioners in this field. Exemplary subjects include human beings of any age with risk factors for cardiovascular disease. Common risk factors include, but are not limited to, age, sex, weight, family history, sleep apnea, alcohol or tobacco use, physical inactivity, arrhythmia, or signs of insulin resistance such as acanthosis nigricans, hypertension, dyslipidemia, or polycystic ovary syndrome (PCOS).

“Treating” or “treatment” cover the treatment of a disease-state as understood by practitioners in this field and include the following: (a) inhibiting the disease-state, i.e., arresting it development; (b) relieving the disease-state, i.e., causing regression of the disease state; and/or (c) preventing the disease-state from occurring in a mammal, in particular, when such mammal is predisposed to the disease-state but has not yet been diagnosed as having it.

“Preventing” or “prevention” cover the preventive treatment (i.e., prophylaxis and/or risk reduction) of a subclinical disease-state aimed at reducing the probability of the occurrence of a clinical disease-state as understood by practitioners in this field. Patients are selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state. “Risk reduction” or “reducing risk” covers therapies that lower the incidence of development of a clinical disease state. As such, primary and secondary prevention therapies are examples of risk reduction.

“Therapeutically effective amount” is intended to include an amount of a compound of the present invention that is effective when administered alone or in combination with other agents to treat disorders as understood by practitioners in this field. When applied to a combination, the term refers to combined amounts of the active ingredients that result in the preventive or therapeutic effect, whether administered in combination, serially, or simultaneously.

“Disorders of the cardiovascular system” or “cardiovascular disorders” include for example the following disorders: hypertension (high blood pressure), peripheral and cardiac vascular disorders, coronary heart disease, stable and unstable angina pectoris, heart attack, myocardial insufficiency, abnormal heart rhythms (or anbythmias), persistent ischemic dysfunction (“hibernating myocardium”), temporary postischemic dysfunction (“stunned myocardium”), heart failure, disturbances of peripheral blood flow, acute coronary syndrome, heart failure, heart muscle disease (cardiomyopathy), myocardial infarction and vascular disease (blood vessel disease).

“Heart failure” includes both acute and chronic manifestations of heart failure, as well as more specific or related types of disease, such as advanced heart failure, post-acute heart failure, cardio-renal syndrome, heart failure with impaired kidney function, chronic heart failure, chronic heart failure with mid-range ejection fraction (HFmEF), compensated heart failure, decompensated heart failure, right heart failure, left heart failure, global failure, ischemic cardiomyopathy, dilated cardiomyopathy, heart failure associated with congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, heart failure associated with combined heart valve defects, myocardial inflammation (myocarditis), chronic myocarditis, acute myocarditis, viral myocarditis, diabetic heart failure, alcoholic cardiomyopathy, heart failure associated with cardiac storage disorders, diastolic heart failure, systolic heart failure, acute phases of worsening heart failure, heart failure with preserved ejection fraction (HFpEF), heart failure with reduced ejection fraction (HFrEF), chronic heart failure with reduced ejection fraction (HFrEF), chronic heart failure with preserved ejection fraction (HFpEF), post myocardial remodeling, angina, hypertension, pulmonary hypertension and pulmonary artery hypertension.

“Fibrotic disorders” encompasses diseases and disorders characterized by fibrosis, including among others the following diseases and disorders: hepatic fibrosis, cirrhosis of the liver, NASH, pulmonary fibrosis or lung fibrosis, cardiac fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitreoretinopathy and disorders of the connective tissue (for example sarcoidosis).

Relaxin-associated disorders include but are not limited to disorders of the cardiovascular system and fibrotic disorders.

The compounds of this invention can be administered by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

“Pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, anti-bacterial agents, anti-fungal agents, lubricating agents and dispensing agents, depending on the nature of the mode of administration and dosage forms.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example. Allen. L. V., Jr, et al., Remington: The Science and Practice of Pharmacy (2 Volumes), 22nd Edition. Pharmaceutical Press (2012).

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.01 to about 5000 mg per day, preferably between about 0.1 to about 1000 mg per day, and most preferably between about 0.1 to about 250 mg per day. Intravenously, the most preferred doses will range from about 0.01 to about 10 mg/kg/minute during a constant rate infusion. Compounds of this invention may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 2000 milligrams of active ingredient per dosage unit. In these pharmaceutical compositions the active ingredient will ordinarily be present in an amount of about 0.1-95% by weight based on the total weight of the composition. A typical capsule for oral administration contains at least one of the compounds of the present invention (250 mg), lactose (75 mg), and magnesium stearate (15 mg). The mixture is passed through a 60 mesh sieve and packed into a No. 1 gelatin capsule. A typical injectable preparation is produced by aseptically placing at least one of the compounds of the present invention (250 mg) into a vial, aseptically freeze-drying and sealing. For use, the contents of the vial are mixed with 2 mL of physiological saline, to produce an injectable preparation.

The compounds may be employed in combination with other suitable therapeutic agents useful in the treatment of diseases or disorders including: anti-atherosclerotic agents, anti-dyslipidemic agents, anti-diabetic agents, anti-hyperglycemic agents, anti-hyperinsulinemie agents, anti-thrombotic agents, anti-retinopathie agents, anti-neuropathic agents, anti-nephropathic agents, anti-ischemic agents, anti-hypertensive agents, anti-obesity agents, anti-hyperlipidemie agents, anti-hypertriglyceridemic agents, anti-hypercholesterolemie agents, anti-restenotic agents, anti-pancreatic agents, lipid lowering agents, anorectic agents, memory enhancing agents, anti-dementia agents, cognition promoting agents, appetite suppressants, agents for treating heart failure, agents for treating peripheral arterial disease, agents for treating malignant tumors, and anti-inflammatory agents.

The additional therapeutic agents may include ACE inhibitors, β-blockers, diuretics, mineralocorticoid receptor antagonists, ryanodine receptor modulators, SERCA2a activators, renin inhibitors, calcium channel blockers, adenosine A1 receptor agonists, partial adenosine A1 receptor, dopamine β-hydroxylase inhibitors, angiotensin II receptor antagonists, angiotensin II receptor antagonists with biased agonism for select cell signaling pathways, combinations of angiotensin II receptor antagonists and neprilysin enzyme inhibitors, neprilysin enzyme inhibitors, soluble guanylate cyclase activators, myosin ATPase activators, rho-kinase 1 inhibitors, rho-kinase 2 inhibitors, apelin receptor agonists, nitroxyl donating compounds, calcium-dependent kinase II inhibitors, antifibrotic agents, galectin-3 inhibitors, vasopressin receptor antagonists, FPR2 receptor modulators, natriuretic peptide receptor agonists, transient receptor potential vanilloid-4 channel blockers, anti-arrhythmic agents, I_{f} “funny current” channel blockers, nitrates, digitalis compounds, inotropic agents and β-receptor agonists, cell membrane resealing agents for example Poloxamer 188, anti-hyperlipidemic agents, plasma HDL-raising agents, anti-hypercholesterolemic agents, cholesterol biosynthesis inhibitors (such as HMG CoA reductase inhibitors), LXR agonist, FXR agonist, probucol, raloxifene, nicotinic acid, niacinamide, cholesterol absorption inhibitors, bile acid sequestrants, anion exchange resins, quaternary amines, cholestyramine, colestipol, low density lipoprotein receptor inducers, clofibrate, fenofibrate, bezafibrate, ciprofibrate, gemfibrizol, vitamin B6, vitamin B12, anti-oxidant vitamins, anti-diabetes agents, platelet aggregation inhibitors, fibrinogen receptor antagonists, aspirin and fibric acid derivatives, PCSK9 inhibitors, aspirin, and P2Y12 Inhibitors such as Clopidogrel.

The additional therapeutic agents may also include nintedanib, Pirfenidone, LPA1 antagonists, LPA1 receptor antagonists, GLP1 analogs, tralokinumab (IL-13, AstraZeneca), vismodegib (hedgehog antagonist, Roche), PRM-151 (pentraxin-2, TGF beta-1, Promedior), SAR-156597 (bispecific Mab IL-4&IL-13, Sanofi), simtuzumab ((anti-lysyl oxidase-like 2 (anti-LOXL2) antibody, Gilead), CKD-942, PTL-202 (PDE inh./pentoxifylline/NAC oral control. release, Pacific Ther.), omipalisib (oral PI3K/mTOR inhibitor. GSK), IW-001 (oral sol, bovine type V collagen mod., Immune Works), STX-100 (integrin alpha V/beta-6 ant, Stromedix/Biogen), Actimmune (IFN gamma), PC-SOD (midismase; inhaled, LTT Bio-Pharma/CKD Pharm), lebrikizumab (anti-IL-13 SC humanized mAb, Roche), AQX-1125 (SHIP I activator, Aquinox), CC-539 (JNK inhibitor, Celgene), FG-3019 (FibroGen), SAR-100842 (Sanofi), and obeticholic acid (OCA or INT-747, Intercept).

The above other therapeutic agents, when employed in combination with the compounds of the present invention may be used, for example, in those amounts indicated in the Physicians' Desk Reference, as in the patents set out above, or as otherwise determined by practitioners in the art.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

The compounds of the present invention are also useful as standard or reference compounds, for example as a quality standard or control, in tests or assays involving RXFP1. Such compounds may be provided in a commercial kit, for example, for use in pharmaceutical research involving RXFP1. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness. The compounds of the present invention may also be used in diagnostic assays involving RXFP1.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises a first therapeutic agent, comprising a compound of the present invention or a pharmaceutically acceptable salt form thereof; and, (c) a package insert stating that the pharmaceutical composition can be used for the treatment of dyslipidemias and the sequelae thereof. In another embodiment, the package insert states that the pharmaceutical composition can be used in combination (as defined previously) with a second therapeutic agent for the treatment of dyslipidemias and the sequelae thereof. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to bold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### Chemical Methods

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene, T. W, et al., Protecting Groups in Organic Synthesis, 4th Edition, Wiley (2007)).

Abbreviations are defined as follows: “1×” for once, “2×” for twice, “3×” for thrice, “° C.” for degrees Celsius, “aq” for aqueous, “eq” or “equiv.” for equivalent or equivalents, “g” for gram or grams, “mg” for milligram or milligrams. “L” for liter or liters, “mL” for milliliter or milliliters, “μL” for microliter or microliters, “N” for normal, “M” for molar, “nM” for nanomolar, “pM” for picomolar, “mol” for mole or moles, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “aq” for “aqueous”. “sat.” for saturated, “MW” for molecular weight, “MS” or “Mass Spec” for mass spectrometry, “ESI” for electrospray ionization mass spectroscopy, “LC-MS” for liquid chromatography mass spectrometry, “HPLC” for high pressure liquid chromatography. “RP HPLC” for reverse phase HPLC, “NMR” for nuclear magnetic resonance spectroscopy, “SFC” for super critical fluid chromatography, “¹H” for proton, “δ” for delta, “s” for singlet, “d” for doublet, “t” for triplet, “q” for quartet, “m” for multiplet, “br” for broad, “Hz” for hertz, “MHz” for megahertz, and “α”, “β”, “R”, “S”, “E”, and “Z” are stereochemical designations familiar to one skilled in the art.

| | |
|---|---|
| AcCl | acetyl chloride |
| AcOH | acetic acid |
| AIBN | Azobisisobutyronitrile |
| BHFFT | bis(tetramethylene)fluoroformadmidinium |
| | hexafluorophosphate |
| Boc | tert-butyloxycarbonyl |
| BuLi | butyl lithium |
| DAST | Diethylaminosulfur trifluoroide |
| DCE | Dichloroethane |
| DCM | Dichloromethane |
| DIEA | diispropyl ethylamine |
| DMAP | 4-dimethylamino pyridine |
| DMF | Dimethylformamide |
| DPPA | Diphenyl phosphorylazide |
| Et₂O | diethyl ether |
| EtOAc | Ethylacetate |
| EtOH | Ethanol |
| HATU | (1-[Bis(dimethylamino)methylene]-1H-1,2,3-triazolo |
| | [4,5-b]pyridinium 3-oxid hexafluorophosphate) |
| HMPA | hexamethylphosphoramide |
| IPA | isopropanol |
| i-Pr | Isopropyl |
| KHMDS | potassium bis(trimethylsilyl)amidel |
| LDA | lithium diisopropyl amide |
| MeCN | Acetonitrile |
| MeOH | Methanol |
| Me | Methyl |
| NBS | N-bromosuccinimide |
| Pd/C | palladium on carbon |
| pTsOH | p-toluenesulfonic acid |
| PyBroP | Bromotripyrrolidinophosphonium hexafluorophosphate |
| T3P | 2,4,6-Tripropyl-1,3,5,2,4,6-trioxatriphosphorinane-2,4,6- |
| | trioxide |
| TBAF | tetra-n-butyl ammonium fluoride |
| t-Bu | tert-butyl |
| Teoc | 2-(trimethylsilyl)ethyl carboxylate |
| TFA | trifluoro acetic acid |
| TFAA | trifluoro acetic anhydride |
| THF | Tetrahydrofuran |
| TsOH | Tolulenesulfonic acid |
| XPhos- | 2nd generation XPhos precatalyst CAS no. 1310584-14-5 |
| Pd-G2 | |

The following methods were used in the exemplified examples, except where noted otherwise. Purification of intermediates and final products was carried out via either normal or reverse phase chromatography. Normal phase chromatography was carried out using prepacked SiO₂ cartridges eluting with either gradients of hexanes and ethyl acetate or DCM and MeOH unless otherwise indicated. Reverse phase preparative HPLC was carried out using C18 columns with UV 220 nm or prep LCMS detection eluting with gradients of Solvent A (90% water, 10% MeOH, 0.1% TFA) and Solvent B (10% water, 90% MeOH, 0.1% TFA) or with gradients of Solvent A (95% water, 5% ACN, 0.1% TFA) and Solvent B (5% water, 95% ACN, 0.1% TFA) or with gradients of Solvent A (95% water, 2% ACN, 0.1% HCOOH) and Solvent B (98% ACN, 2% water, 0.1% HCOOH) or with gradients of Solvent A (95% water, 5% ACN, 10 mM NH₄OAc) and Solvent B (98% ACN, 2% water, 10 mM NH₄OAc) or with gradients of Solvent A (98% water, 2% ACN, 0.1% NH₄OH) and Solvent B (98% ACN, 2% water, 0.1% NH₄OH).

LC/MS methods employed in characterization of examples are listed below.

Analytical HPLC Method A conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

Analytical HPLC Method B conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm).

Analytical HPLC Method C conditions: Column: Sunfire C18, 3.0×150 mm, 3.5 μm particles, Mobile Phase A: 5:95 acetonitrile:water with 0.05% trifluoroacetic acid; Mobile Phase B: 95:5 acetonitrile:water with 0.05% trifluoroacetic acid; Gradient: 0% B to 100% B over 12 minutes; Flow: 0.5 mL/min; Detection: UV (220 nM and 254 nM). NMR Employed in Characterization of Examples. ¹H NMR spectra were obtained with Bruker Fourier transform spectrometers operating at frequencies as follows: 1H NMR: 400 MHz (Bruker) or 500 MHz (Bruker). Spectra data are reported in the format: chemical shift (multiplicity, coupling constants, number of hydrogens). Chemical shifts are specified in ppm downfield of a tetramethylsilane internal standard (8 units, tetramethylsilane=0 ppm) and/or referenced to solvent peaks, which in ¹H NMR spectra appear at 2.51 ppm for DMSO-d₆, 3.30 ppm for CD₃OD, 1.94 ppm for CD₃CN, and 7.24 ppm for CDCl₃.

Scheme I shows how compounds of the invention may be prepared from racemic intermediate I-1, which is a known compound, the synthesis of which is described in Org. Lett. 2012, 14, 4, 1110-1113. Hydrogenolysis of the benzyl ester in I-1 yields methyl ester/carboxylic acid I-2. The amine in I-3 is installed as a Teoc carbamate via a Curtius rearragement. Hydrolysis of the remaining ester and coupling with an amine or aniline yields 1-4. Cleavage of the carbamate under acidic conditions such as HCl in dioxane or TFA in CH₂Cl₂ yields an amine suitable for coupling with a variety of carboxylic acids (1-5) using standard amine coupling reagents such as HATU or BOP to furnish Examples I.

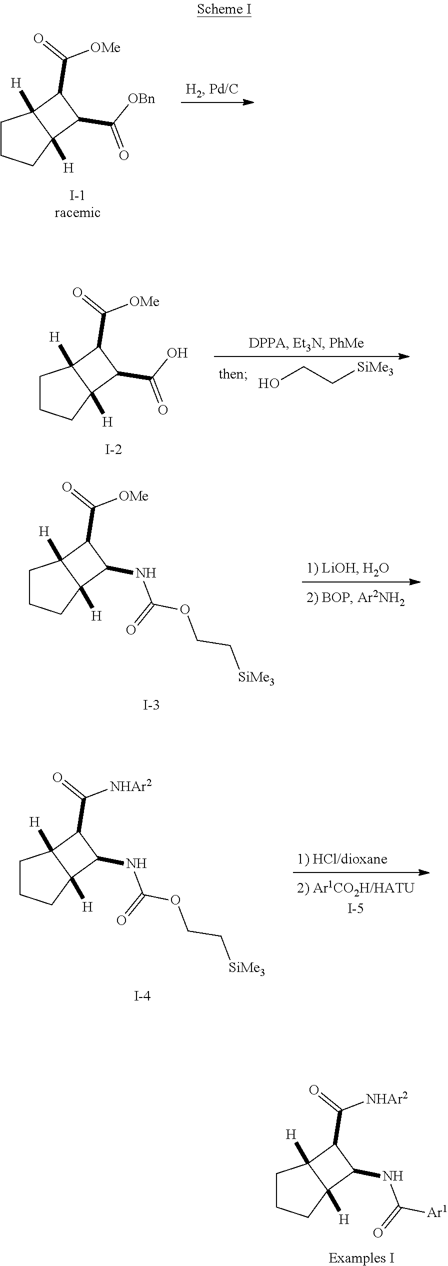

Scheme II illustrates a general route to mandelic acid-based biaryl analogs. Commercially available II-1 was converted to the t-butyl ester II-2, then brominated to furnish II-3. Displacement of the bromide with acetic acid furnished intermediate II-4, which was then subjected to a Suzuki reaction to furnish Intermediate 8 (acetate cleavage was concomitant with biaryl formation). The resulting acid was directly coupled to an amine intermediate as was described in Scheme I to furnish II-6. The hydroxyl group in II-6 could be elaborated with either the appropriate isocyanate or a two-step carbamate forming protocol (e.g., nitrophenyl chloroformate, TEA, followed by an amine). The t-butyl ester is then cleaved (TFA/DCM) to furnish compounds of this invention.

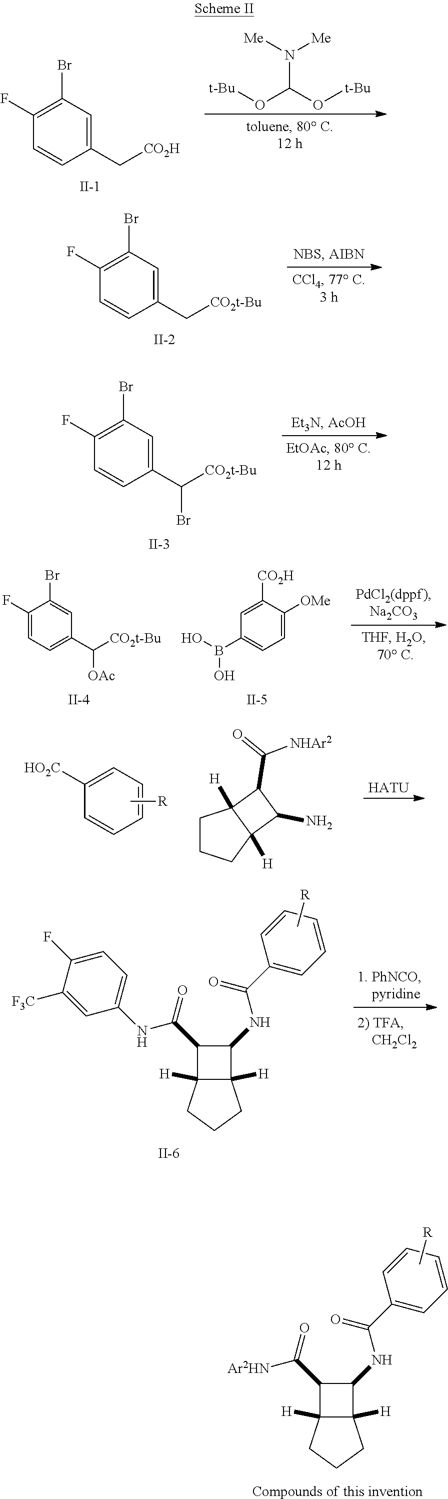

Scheme III describes a route for the production of substituted isoxazoline analogs. Treatment of III-1 with NaOCl, followed by a substituted olefin with subsequent saponification of the ester provided intermediates III-2. These intermediates were coupled with amines according to the methods outlined in Scheme I to furnish compounds of this invention.

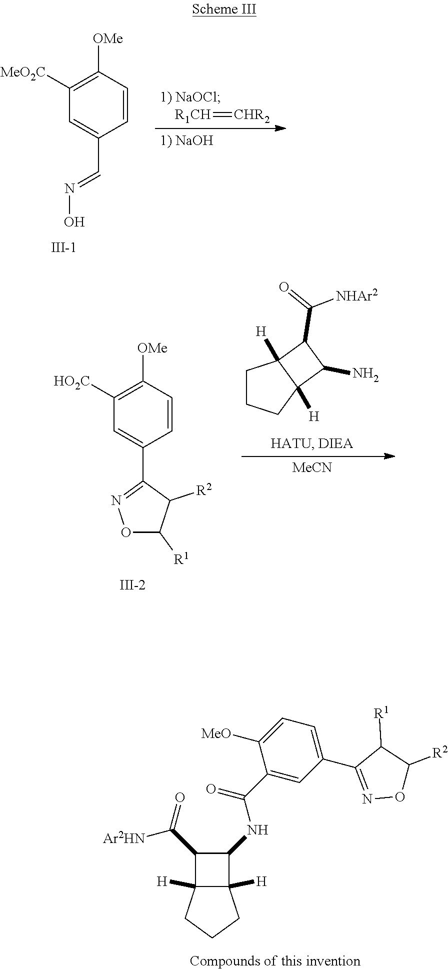

Scheme IV describes the photochemical [2+2] reaction to generate the core structure of the benzo-fused examples of this invention. Reaction of indene IV-1 with mono-benzyl maleate IV-2 in the presence of an appropriate photocatalyst such as (Ir[dF(CF₃)ppy]₂ (dtbpy))PF₆ upon exposure to blue or purple LED lights yields [2+2] adducts IV-3 and IV-4 (benzo-fusion regioisomers resulting from endo cycloaddition), IV-5 and IV-6 (benzo-fusion regioisomers resulting from exo cycloaddition) and IV-7, IV-8, IV-9, and IV-10 (the trans-isomers of IV-3 to IV-6).

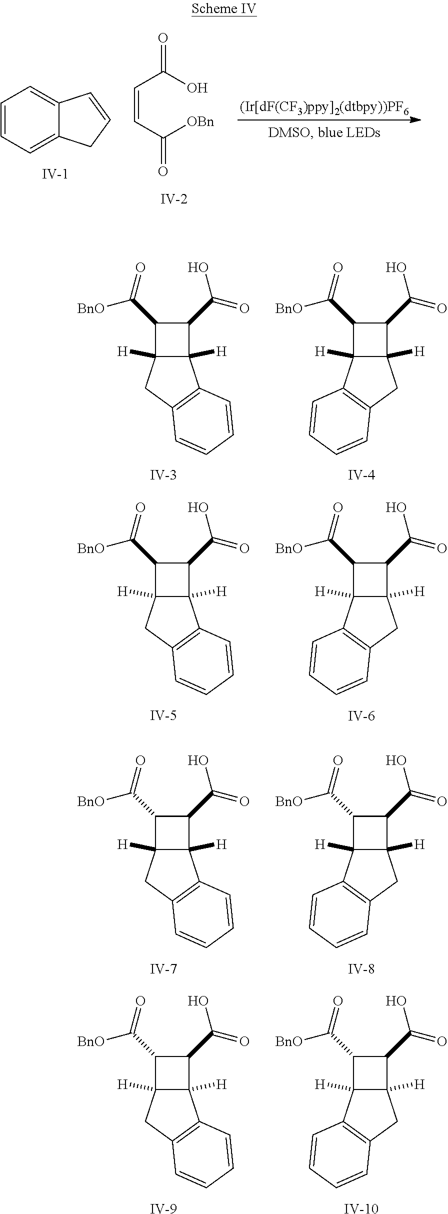

Scheme V describes the synthesis of benzo-fused analogs from the intermediates in Scheme IV, using IV-3 as an example, but not limited to. IV-3 can be subjected to a Curtius rearragement to yield V-1. Hydrogenolysis of the remaining benzyl ester and standard amine coupling conditions with an appropriate amine V-2 affords V-3. Cleavage of the carbamate in V-3 under acidic conditions such as HCl in dioxane or TFA in CH₂Cl₂ yields an amine suitable for coupling with a variety of carboxylic acids (V-4) using standard amine coupling reagents such as HATU or BOP to furnish compounds of the general structure, V-5.

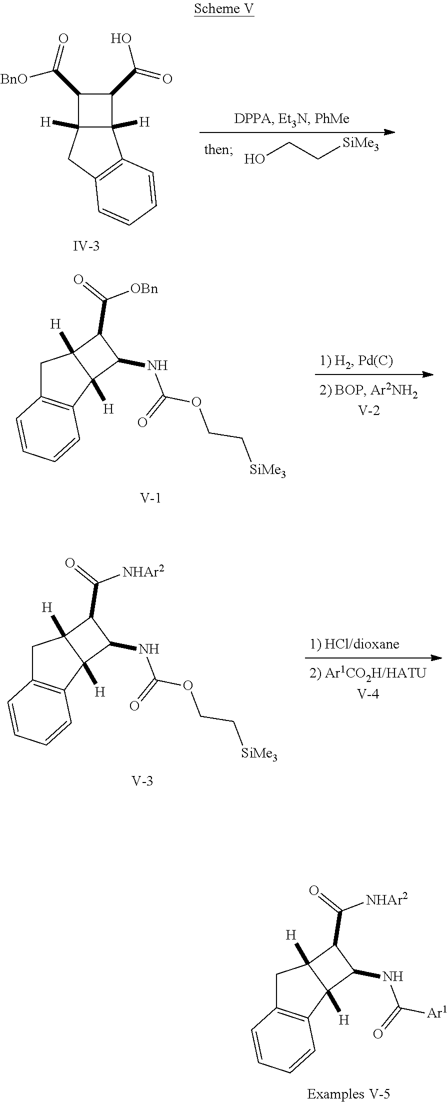

### Intermediate 1-cis-6-benzyl 7-methyl-bicyclo[3.2.0]heptane-6,7-dicarboxylate

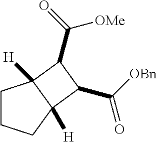 [SMILES: COC(=O)[C@@H]1[C@H]2CCC[C@H]2[C@@H]1C(=O)OCc1ccccc1]

Intermediate 1 was prepared as a racemate by using the methods described in Org. Lett. 2012, 14, 4, 1110-1113.

### Intermediate 2-5-(3-hydroxyprop-1-yn-1-yl)-2-methoxybenzoic acid

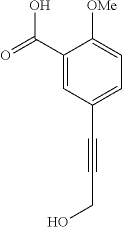 [SMILES: COc1ccc(C#CCO)cc1C(=O)O]

### Intermediate 2A—Preparation of methyl 5-(3-hydroxyprop-1-yn-1-yl)-2-methoxybenzoate

To methyl 5-bromo-2-methoxybenzoate (3.0 g, 12 mmol) and propargyl alcohol (1.5 mL, 25 mmol) slurried in TEA (31 mL) was added Pd(PPh₃)₄ (0.28 g, 0.25 mmol) and CuI (0.023 g, 0.12 mmol). The vessel was sparged with N₂ and heated at 80° C. for 16 h. The reaction mixture was partitioned between water with EtOAc and the organic layer was separated and dried over Na₂SO₄. The organic layer was decanted and concentrated under vacuum and the residue loaded onto an 80 g Isco column and eluted with a linear gradient of 0% to 100% EtOAc in hexanes to furnish methyl 5-(3-hydroxyprop-1-yn-1-yl)-2-methoxybenzoate (1.3 g, 6.1 mmol, 50% yield). ¹H NMR (500 MHz, CDCl₃) δ 7.92 (d, J=2.1 Hz, 1H), 7.56 (dd, J=8.7, 2.3 Hz, 1H), 6.95 (d, J=8.7 Hz, 1H), 4.50 (d, J=6.1 Hz, 2H), 3.94 (s, 3H), 3.91 (s, 3H), 1.64 (t, J=6.1 Hz, 1H), MS (ESI) m/z 221.1 (M+H)⁺.

Intermediate 2: Into a 2 dram reaction vial was added Intermediate 2A (50 mg, 0.23 mmol), THF (1 mL), water (0.5 mL), and LiOH monohydrate (35 mg, 0.85 mmol). The reaction mixture was stirred at 23° C. for 1 h, diluted with EtOAc (10 mL), and washed with sat. NH₄Cl containing 1.5 mmol HCl. The organic phase was dried over Na₂SO₄, filtered, and concentrated under reduced pressure to provide Intermediate 2 that was used without further purification (47 mg, 0.23 mmol) LC-MS RT: 0.76 min; MS (ESI) m/z=206.8 (M+H)⁺.

### Intermediate 3 (chiral peak-1), Intermediate 4 (chiral peak-2): 2-methoxy-5-(3a,4,6,6a-tetrahydrofuro[3,4-d]isoxazol-3-yl)benzoic acid

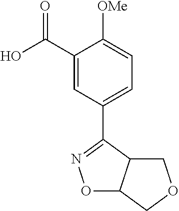 [SMILES: COc1ccc(C2=NOC3COCC23)cc1C(=O)O]

Intermediate 3A: Preparation of methyl (E)-5-((hydroxyimino)methyl)-2-methoxybenzoate: Commercially available methyl 5-formyl-2-methoxybenzoate (1.16 g, 6.00 mmol) was dissolved in DCM (5 mL), and to this solution was added HONH₂·HCl (415 mg, 6.00 mmol) followed by TEA (1 mL) and the reaction mixture was stirred at room temperature for 20 hours. The reaction mixture was diluted with water (100 mL) and extracted with EtOAc (2×25 mL). The combined organic layers were dried with MgSO₄ and concentrated to dryness in vacuo to afford Intermediate 3A as a white solid (1.2 g, 95%). ¹H NMR (400 MHZ, CDCl₃) δ 8.13 (s, 1H), 8.03 (d, J=2.4 Hz, 1H), 7.78-7.67 (m, 1H), 7.03 (d, J=8.8 Hz, 1H), 3.97 (s, 3H), 3.93 (s, 3H). LCMS (ESI) m/z: =210.1 (M+H)⁺.

Intermediate 3B: Preparation of methyl (Z)-5-(chloro(hydroxyimino)methyl)-2-methoxybenzoate: Intermediate 3A (23 g, 0.10 mol) was dissolved in DMF (100 mL), and to this solution was added NCS (15 g, 0.10 mol) and the reaction mixture was stirred at room temperature for 24 hours. The reaction was quenched with water (300 mL), at which point a solid formed, which was collected by filtration. The filtered solid was then dried in vacuo to afford Intermediate 3B (23 g, 86% yield) as a pale yellow solid. LCMS (ESI) m/z: =244.1 (M+H)⁺.

Intermediate 3C: Preparation of methyl 2-methoxy-5-(3a,4,6,6a-tetrahydrofuro[3,4-d]isoxazol-3-yl)benzoate: To intermediate Intermediate 3B (0.4 g, 2 mmol) and 2,5-dihydrofuran (1.2 g, 17 mmol) in DCM (10 mL) was added TEA (0.7 mL, 5.2 mmol) and the reaction mixture was stirred 24 h. The solvents were removed under reduced pressure and the residue was loaded onto a 40 g Isco column and eluted with a linear gradient of 0% to 10% MeOH in CH₂Cl₂ to afford Intermediate 3C (0.40 g, 83% yield). ¹H NMR (500 MHz, CDCl₃) δ 7.98 (d, J=2.3 Hz, 1H), 7.86 (dd, J=8.8, 2.4 Hz, 1H), 7.04 (d, J=8.7 Hz, 1H), 5.38 (dd, J=9.2, 3.9 Hz, 1H), 4.34-4.26 (m, 2H), 4.20-4.09 (m, 1H), 3.96 (s, 3H), 3.91 (s, 3H), 3.83-3.76 (m, 1H), 2.92-2.70 (m, 1H). LCMS (ESI) m z=278.3 (M+H)⁺.

Chiral Intermediate 3D and 3E: The following chiral intermediates were separated by chiral SFC by the following preparative chromatographic methods from Intermediate 3C: Instrument: Berger MG II Column: Chiralpak IA, 21×250 mm, 5 micron, Mobile Phase: 20% MeOH/80% CO₂. Flow Conditions: 45 mL/min, 150 Bar, 40° C., Detector Wavelength: 220 nm; Analytical method: Instrument: Shimadzu Nexera SFC, Column Chiralpak IA, 4.6×100 mm, 3 micron, Mobile Phase: 20% MeOH/80% CO₂. Flow Conditions: 2 mL/min, 150 Bar, 40° C., Detector Wavelength: 220 nm, to afford chiral Intermediate 3D (Peak-1, RT=3.80 min., >99% ee) and chiral Intermediate 3E (Peak-2, RT=7.43 min., >98% ee).

Intermediate 3: Preparation of 2-methoxy-5-(3a,4,6,6a-tetrahydrofuro[3,4-d]isoxazol-3-yl)benzoic acid: To a solution of Intermediate 3D (75 mg, 0.30 mmol) in THF (3 mL) was added MeOH (0.6 mL) followed by LiOH (2M, 0.4 mL, 0.8 mmol). After 4 hours, the reaction mixture was diluted with water (20 mL) and the solution was brought to pH=4-5 with HCl (1N). The reaction mixture was extracted with EtOAc (2×25 mL) and the combined organic layers were dried (MgSO₄) and concentrated to dryness under reduced pressure to afford Intermediate 3 (71 mg, 100% yield). ¹H NMR (400 MHZ, CD₃OD) δ 7.78 (d, J=2.3 Hz, 1H), 7.71 (dd. J=8.7, 2.3 Hz, 1H), 7.09 (d, J=8.8 Hz, 1H), 5.33 (dd, J=9.2, 3.6 Hz, 1H), 4.44 (ddd, J=8.9, 7.2, 1.3 Hz, IH), 4.18 (d, J=10.6 Hz, 1H), 4.08 (dd, J=9.4, 1.0 Hz, 1H), 3.91-3.88 (m, 3H), 3.86 (dd, J=9.4, 6.9 Hz, 1H), 3.73 (dd, J=10.7, 3.7 Hz, 1H). LCMS (ESI) m/z: =264.1 (M+H)⁺.

Intermediate 4: Preparation of 2-methoxy-5-(3a,4,6,6a-tetrahydrofuro[3,4-d]isoxazol-3-yl)benzoic acid: Intermediate 3E (52 mg, 0.2 mmol, 100% yield) was prepared in a similar manner to Intermediate 3,

substituting Intermediate 3E for Intermediate 3D. ¹H NMR (500 MHz, CD₃OD) δ 8.10 (d, J=2.4 Hz, 1H), 7.87 (dd, J=8.8, 2.4 Hz, 1H), 7.23 (d, J=8.7 Hz, 1H), 5.38 (dd, J=9.2, 3.7 Hz, 1H), 4.47 (ddd, J=8.9, 7.2, 1.2 Hz, 1H), 4.22 (d, J=10.7 Hz, 1H), 4.12 (q, J=7.1 Hz, 1H), 4.07 (dd, J=9.5, 0.9 Hz, 1H), 3.97 (s, 3H), 3.88 (dd, J=9.5, 6.9 Hz, 1H), 3.76 (dd. J=10.8, 3.6 Hz, 1H). LCMS (ESI) m/z: =264.1 (M+H)+.

### Intermediate 5C: 5-(5-hydroxy-3a,5,6,6a-tetrahydro-4H-cyclopenta[d]isoxazol-3-yl)-2-methoxybenzoic acid

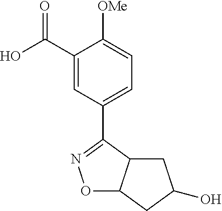 [SMILES: COc1ccc(C2=NOC3CC(O)CC23)cc1C(=O)O]

Intermediate 5 was prepared as a mixture of 4 isomers in a similar manner as Intermediate 3C by substituting cyclopent-3-ene-1-ol for cyclopent-3-en-1-ylmethanol followed by ester hydrolysis as described for Intermediate 3 to afford the diasteromeric mixture of Intermediates 5. ¹H NMR (600 MHz, CDCl₃) δ 8.04 (d, J=2.3 Hz, 1H), 7.85 (dd, J=8.8, 2.3 Hz, 1H), 7.03 (d, J=8.8 Hz, 1H), 5.30 (ddd, J=9.4, 6.2, 2.9 Hz, 1H), 4.50 (quin, J=5.9 Hz, 1H), 4.19 (td, J=9.3, 4.7 Hz, 1H), 3.92 (s, 3H), 2.33-2.27 (m, 1H), 2.18-2.06 (m, 3H). MS (ESI) m/z=292.0 (M+H)⁺.

The isomers of Intermediate 5 were separated by chiral SFC by the following preparative chromatographic methods: Instrument: Berger SFC (LVL-L4021 Lab) Column: IC 25×3 cm ID, 5 μm, Temperature: 40° C., Flow rate: 85 mL/min, Mobile Phase: gradient 75/25 CO₂/MeOH for 12 min then to 45% MeOH, Detector Wavelength: 235 nm, Injection Volume: 1000 μL to afford Intermediate 5A (peak 1, >99% ee, Analytical RT=8.80 min), Intermediate 5B (peak 2, >95% ee, Analytical RT=9.86 min), Intermediate 5C (peak 3, >99% ee, Analytical RT=13.53 min), Intermediate SD (peak 4, >99% ee, Analytical RT=16.67 min). Analytical Chromatographic Conditions: Instrument: Agilent SFC (LVL-L4021 Lab), Column: IC 250×4.6 mm ID, 5 μm, Temperature: Ambient, Flow rate: 2.0 mL/min. Mobile Phase: gradient 75/25 CO₂/MeOH 12 min then to 45% MeOH. Analytical data for peak-1-4: ¹H NMR (600 MHz, CD₃OD) δ 8.07 (d, J=2.2 Hz, 1H), 7.82 (dd, J=8.7, 2.1 Hz, 1H), 7.18 (d, J=8.8 Hz, 1H), 5.21 (ddd, J=9.2, 6.2, 2.5 Hz, 1H), 4.27 (m, 1H), 4.24 (td, J=9.4, 4.0 Hz, 1H), 3.94 (s, 3H), 2.16 (m, 1H), 2.05 (m, 1H), 2.00 (m, 1H), 1.99 (m, 1H), 13C NMR (151 MHz, CD₃OD) δ 169.5, 161.6, 160.0, 133.2, 131.4, 122.6 (2C), 113.9, 87.3, 72.7, 56.8, 51.5, 44.1, 40.3.

### Intermediate 6: (S)-5′-(1-((cyclobutylcarbamoyl)oxy)-2,2,2-trifluoroethyl)-2′-fluoro-4-methoxy-[1,1′-biphenyl]-3-carboxylic acid

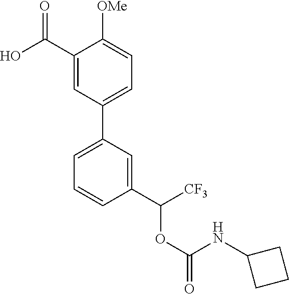 [SMILES: COc1ccc(-c2cccc(C(OC(=O)NC3CCC3)C(F)(F)F)c2)cc1C(=O)O]

Intermediate 6A—Preparation of 1-(3-bromo-4-fluorophenyl)-2,2,2-trifluoroethan-1-ol. 3-Bromo-4-fluorobenzaldehyde (0.2 g, 1.2 mmol) was dissolved in DMF (3.5 mL), and to this solution was added (trifluoromethyl)trimethylsilane (0.30 mL, 2.3 mmol), and K₂CO₃ (8.0 mg, 58 μmol). The reaction mixture was stirred at rt for 60 min and HCl (2N, 3 mL) was added. After stirring at room temperature for an additional 1 hour, the reaction mixture was diluted with EtOAc (15 mL), and the solution washed with sat. NH₄Cl (25 mL). The aqueous phase was extracted with EtOAc (2×10 mL), dried (Na₂SO₄), concentrated under reduced pressure and the residue purified by silica gel chromatography to produce Intermediate 6A (0.2 g, 0.8 mmol, 65% yield). ¹H NMR (500 MHz, CDCl₃) δ 7.74 (dd, J=6.5, 2.1 Hz, 1H), 7.43 (ddd, J=8.4, 4.8, 2.2 Hz, 1H), 7.19 (t, J=8.4 Hz, 1H), 5.11-4.98 (m, 1H), 2.69 (d, J=4.4 Hz, 1H).

Intermediate 6B-Preparation of(S)-1-(3-bromo-4-fluorophenyl)-2,2,2-trifluoroethan-1-ol. (S)-2-phenyl-2,3-dihydrobenzo[d]imidazo[2,1-b]thiazole (0.4 g, 1.6 mmol) and Intermediate 6A (11 g, 40 mmol) were dissolved in diisopropyl ether (134 mL) and chilled between 0° C. to −20° C. The solution was then treated with isobutyl anhydride (4 ml, 24 mmol) and transferred to a freezer (@—20° C.) for 18 h. The reaction was quenched with MeOH (1 mL) with phosphate buffer and the resulting solution extracted with EtOAc (2×25 mL), the organic extracts dried (MgSO₄) concentrated under reduced pressure to a residue which was purified via normal phase chromatography using hexanes/EtOAc as eluents to afford Intermediate 6B (5.0 g, 18 mmol, 44% yield, >99% ee). ¹H NMR (500 MHz, CDCl₃) δ 7.74 (dd, J=6.3, 1.9 Hz, 1H), 7.50-7.39 (m, 1H), 7.18 (t, J=8.4 Hz, 1H), 6.71-5.53 (m, 1H), 5.03 (q, J=6.5 Hz, 1H), Intermediate 6C-Preparation of(S)-1-(3-bromo-4-fluorophenyl)-2,2,2-trifluoroethyl cyclobutylcarbamate. Intermediate 6B (0.30 g, 1.1 mmol), pyridine (0.40 mL, 5.5 mmol), and DMAP (13 mg, 0.10 mmol) were dissolved in DCM (20 mL) and 4-nitrophenyl carbonochloridate (1.0 g, 5.5 mmol) was added. The reaction mixture was stirred for 1 h, followed by the addition of cyclobutanamine (0.78 g, 11 mmol). After 2 h. the reaction mixture was concentrated under reduced pressure and the residue purified by normal phase chromatography using hexanes/EtOAc as eluents to afford Intermediate 6C (0.35 g, 0.90 mmol, 85% yield) as a white solid. LCMS (ESI) m/z 369.7-371.7 (M+H)⁺.

Intermediate 6-Preparation of(S)-5′-(1-((cyclobutylcarbamoyl)oxy)-2,2,2-trifluoroethyl)-2′-fluoro-4-methoxy-[1,1′-biphenyl]-3-carboxylic acid. In a sealed vial was added Intermediate 6C (0.3 g, 0.8 mmol), 5-borono-2-methoxybenzoic acid (0.2 g, 1 mmol), PdCl₂(dppf)-DCM (98 mg, 0.12 mmol), Na₂CO₃ (0.3 g, 3.2 mmol), THF (12 mL) and H₂O (2.9 mL). The reaction mixture was degassed by bubbling N₂ for 10 min, sealed, and stirred at 65° C. for 3 h. After cooling to rt the reaction was quenched with HCl (1N), and extracted with EtOAc (2×25 mL). The combined organic extracts were dried (Na₂SO₄), concentrated under reduced pressure and purified by reverse phase chromatography to afford Intermediate 6 (72 mg, 0.16 mmol, 20% yield). LCMS (ESI) m z 442.0 (M+H)⁺.

### Intermediate 8: 5′-(2-(tert-butoxy)-1-hydroxy-2-oxoethyl)-2′-fluoro-4-methoxy-[1,1′-biphenyl]-3-carboxylic acid

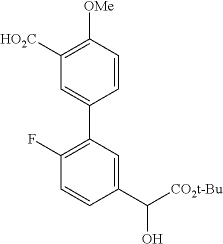 [SMILES: COc1ccc(-c2cc(C(O)C(=O)OC(C)(C)C)ccc2F)cc1C(=O)O]

Intermediate 8A: Intermediate 8A was prepared employing known conditions for analogous substrates (Ludwig. J.; Lehr, M. Syn. Comm. 2004, 34, 3691-3695), except the reaction temperature was maintained at 80° C. for 12 h. ¹H NMR (500 MHz, CDCl₃) δ 7.49 (dd, J=6.6, 2.2 Hz, 1H), 7.20 (ddd, J=8.3, 4.6, 2.2 Hz, 1H), 7.13-7.03 (m, 1H), 3.49 (s, 2H), 1.46 (s, 9H).

Intermediate 8B: To a 20 mL reaction vial charged with Intermediate 8A (266 mg, 0.920 mmol) was added NBS (196 mg, 1.10 mmol), carbon tetrachloride (10 mL), and AIBN (15 mg, 0.090 mmol). The solution was stirred at 77° C., for 3 h, allowed to cool to rt, concentrated under reduced pressure and purified by normal phase silica gel chromatography to give Intermediate 8B (308 mg, 0.840 mmol, 91.0% yield).

Intermediate 8C: To a 2 dram vial charged with Intermediate 8B was added ethyl acetate (2 mL), triethyl amine (0.27 mL, 2.0 mmol), and acetic acid (0.1 mL, 2 mmol). The reaction mixture was stirred at 80° C. for 12 h. The reaction mixture was concentrated under reduced pressure and purified by normal phase silica gel chromatography to give Intermediate 8C. ¹H NMR (500 MHz, CDCl₃) δ 7.70 (dd, J=6.6, 2.2 Hz, 1H), 7.41 (ddd. J=8.4, 4.7, 2.1 Hz, 1H), 7.15 (t. J=8.4 Hz, 1H), 5.77 (s, 1H), 2.22 (s, 3H), 1.43 (s, 9H).

Intermediate 8: Into the reaction vessel containing Intermediate 8C (51 mg, 0.15 mmol) was added 5-borono-2-methoxybenzoic acid (37 mg, 0.19 mmol), PdCl₂ (dppf)-CH₂Cl₂ adduct (24 mg, 0.030 mmol), and Na₂CO₃ (93 mg, 0.88 mmol). The reaction mixture was degassed by bubbling nitrogen for 3 min, sealed, and stirred at 65° C. for 2 h. After allowing to cool to 23° C., the reaction mixture was extracted with EtOAc. The organic phase was dried over Na₂SO₄, filtered, concentrated under reduced pressure, and purified via silica gel chromatography to produce Intermediate 8. Half of the material was isolated as the O-acetate (85 mg, 0.60 mmol, 34%); ¹H NMR (500 MHZ, CDCl₃) δ 8.43-8.36 (m, 1H), 7.81 (dt, J=8.7, 2.0 Hz, 1H), 7.56 (dd, J=7.3, 2.3 Hz, 1H), 7.45 (ddd, J=8.5, 4.6, 2.3 Hz, 1H), 7.23-7.16 (m, 2H), 5.84 (s, 1H), 4.17 (s, 3H), 2.23 (s, 3H), 1.45 (s, 9H) while the other half was isolated as the free alcohol (70 mg, 0.19 mmol, 31%); ¹H NMR (500 MHz, CDCl₃) δ 8.40 (d, J=2.2 Hz, 1H), 7.82 (dt, J=8.6, 2.2 Hz, 1H), 7.54 (dd, J=7.4, 2.5 Hz, 1H), 7.41 (ddd, J=8.4, 4.8, 2.2 Hz, 1H), 7.19-7.14 (m, 2H), 5.09 (s, 1H), 4.16 (s, 3H), 1.47 (s, 9H). Racemic Intermediate 8 was separated into individual enantiomers using chiral SFC. Preparative chromatographic conditions: Instrument: Berger MG II; Column: Chiralpak ID, 21×250 mm, 5 micron; Mobile phase: 25% IPA/75% CO₂; Flow conditions: 45 mL/min, 120 Bar, 40° C.; Detector wavelength: 220 nm; Injection details: 8 injections of 0.36 mL of ˜20 mg/mL in IPA. Analytical chromatrographic conditions: Instrument: Waters UPC2 analytical SFC; Column: Chiralpak ID 4.6×100 mm, 3 micron; Mobile phase: 25% IPA/75% CO₂; Flow conditions: 2 mL/min, 150 Bar, 40° C.; Detector wavelength: 220 nm. Peak 1, RT=3.89 min, >99.5% ee: Peak 2, RT=5.44 min, >99.5% ee. Intermediate 8-2 was collected as Peak #2 (31% yield)

### Intermediate 9:5-(5-(hydroxymethyl)-4,5-dihydroisoxazol-3-yl)-2-methoxybenzoic acid

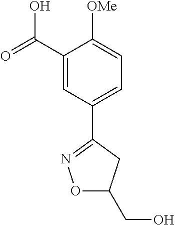 [SMILES: COc1ccc(C2=NOC(CO)C2)cc1C(=O)O]

Intermediate 9A: Preparation of methyl (E)-5-((hydroxyimino)methyl)-2-methoxybenzoate. Commercially available methyl 5-formyl-2-methoxybenzoate (1.16 g, 5.97 mmol) was dissolved in CH: Cl₂ (5 mL), and to this solution was added hydroxylamine HCl (415 mg, 5.97 mmol) followed by Et₃N (1 mL) and the reaction mixture was stirred at rt for 18 h. Water (100 mL) was added and the solution extracted with EtOAc (2×25 mL), the combined organic portions dried (MgSO₄), filtered and evaporated under reduced pressure to generate Intermediate 9A (1.2 g, 95% yield). ¹H NMR (400 MHZ, CDCl₃) δ 8.13 (s, 1H), 8.03 (d, J=2.4 Hz, 1H), 7.78-7.67 (m, 1H), 7.03 (d, J=8.8 Hz, 1H), 3.97 (s, 3H), 3.93 (s, 3H). MS (ESI) m/z=210.1 (M+H)⁺. Intermediate 9B: Preparation of methyl S-(5-(hydroxymethyl)-4,5-dihydroisoxazol-3-yl)-2-methoxybenzoate. Intermediate 9A (55 mg, 0.26 mmol) was dissolved in DMF (2 mL), and to this solution was added NCS (35 mg, 0.26 mmol) and the reaction mixture was stirred at rt for 4 hours. Water (10 mL) was added and the solution extracted with EtOAc (2×25 mL), the combined organic portions were dried (MgSO₄), filtered, concentrated under reduced pressure and the residue immediately re-dissolved in DCM (5 mL). Allyl alcohol (61 mg, 1.1 mmol) was added to the solution followed by TEA (0.5 mL) and the resulting reaction mixture stirred at rt for 18 hours. Water was added (20 mL) and the solution extracted with EtOAc (2×20 mL), the combined organic portions dried (MgSO₄), filtered and purified by normal phase chromatography eluting with hexanes/EtOAc to yield Intermediate 9B (58 mg, 85% yield). ¹H NMR (500 MHZ, CDCl₃) δ 8.05 (d, J=2.4 Hz, 1H), 7.89 (dd, J=8.8, 2.4 Hz, 1H), 7.05 (d, J=8.9 Hz, 1H), 4.90 (dddd, J=10.8, 7.7, 4.6, 3.2 Hz, 1H), 4.08-3.85 (ss, 6H), 3.81-3.68 (m, 1H), 3.46-3.36 (m, 1H), 1.89 (br t, J=6.2 Hz, 1H), 1.57 (s, 2H). MS (ESI) m z=266.1 (M+H)⁺. Intermediate 9C: Intermediate 9B (58 mg, 0.22 mmol) was dissolved in THF (2 mL) and to this was added LiOH (6.3 g, 0.26 mmol) followed by water (2 mL) and methanol (1 mL) and stirred at rt for 4 h. HCl (1N) was added to the reaction mixture to reach pH 7 and the resulting solution extracted with EtOAc (2×25 mL). The combined organic portions were dried (MgSO₄), filtered and concentrated under reduced pressure to afford Intermediate 9C (640 mg, 84% yield). ¹H NMR (500 MHZ, CDCl₃) δ 8.28 (d, J=2.3 Hz, 1H), 8.14 (dd, J=8.8, 2.4 Hz, 1H), 7.28-7.14 (m, 1H), 4.92 (dddd. J=10.8, 7.7, 4.6, 3.1 Hz, 1H), 4.16 (s, 3H), 4.09-3.89 (m, 1H), 3.72 (dd. J=12.4, 4.6 Hz, 1H), 3.48-3.39 (m, 1H), 3.38-3.29 (m, 1H), 1.94-1.72 (m, 1H), 1.60 (br s, 1H). MS (ESI) m/z=252.3 (M+H)⁺.

Intermediates 9D and 9. Intermediate 9C (640 mg) was subjected to chiral SFC separation according to the following preparative method: Instrument: Berger MG II, Column: Chiralpak IC, 21×250 mm, 5 micron Mobile Phase: 20% Methanol/80% CO₂ Flow Conditions: 2 mL/min. 150 Bar, 40° C. Detector Wavelength: 220 nm Injection Details: 0.7 mL of ˜35 mg/mL in MeOH to afford Intermediate 9C (Peak 1, >99% de, Analytical RT=5.6 min) and Intermediate 9 (Peak 2, 99% de, Analytical RT=6.6 min), Analytical Chromatographic Conditions: Instrument: Shimadzu Nexera SFC (CTR-L410-SFC3), Column: Chiralpak IC, 4.6×100 mm, 3 micron, Mobile Phase: 20% Methanol/80% CO₂ Flow Conditions: 2.0 mL/min, 150 Bar, 40° C., Detector Wavelength: 220 nm Injection Details: 5 μL of ˜1 mg/mL in MeOH

### Example 1—6-fluoro-3′-(((1S,5R,6S,7R)-7-((4-fluoro-3-(trifluoromethyl)phenyl)carbamoyl)bicyclo[3.2.0]heptan-6-yl)carbamoyl)-4′-methoxy-[1,1′-biphenyl]-3-carboxylic acid

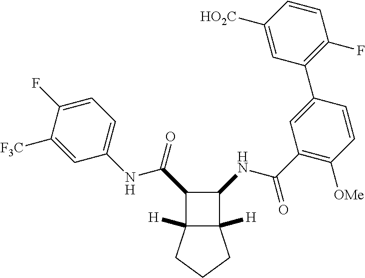 [SMILES: COc1ccc(-c2cc(C(=O)O)ccc2F)cc1C(=O)N[C@H]1[C@@H]2CCC[C@@H]2[C@H]1C(=O)Nc1ccc(F)c(C(F)(F)F)c1]

Example 1-Preparation of methyl (1R,5S,6R,7S)-7-(((2-(trimethylsilyl)ethoxy)carbonyl)amino)bicyclo[3.2.0]heptane-6-carboxylate Intermediate 1 (450 mg, 1.56 mmol) was dissolved in EtOAc (30 mL) in a 250 mL round-bottomed pressure flask and the solution was degassed by nitrogen sparging for 5 minutes. Pd—C(166 mg, 0.156 mmol) was then added under a gentle stream of nitrogen. The flask was then degassed via vacuum/nitrogen back-fill three times and vacuum/hydrogen back-fill two times. The flask was pressurized with 40 psi hydrogen and stirred at room temperature for 3 hours. After depressurizing and back-filling with nitrogen, the reaction mixture was filtered through a plug of Celite® and the filtrate was concentrated in vacuo to yield (1S,5R,6S,7R)-7-(methoxycarbonyl) bicyclo[3.2.0]heptane-6-carboxylic acid which was used without further purification, (300 mg, 1.5 mmol, 97% yield) as a clear, colorless oil. MS (ESI) m/z 199.1 [M+H]⁺. yield (1S,5R,6S,7R)-7-(methoxycarbonyl) bicyclo[3.2.0]heptane-6-carboxylic acid was dissolved in toluene (3 mL) in a 20 mL vial equipped with a Teflon septum cap and Et₃N (420 μl, 3.03 mmol), and DPPA (391 μl, 1.81 mmol) were added. The reaction mixture was stirred at room temperature for 30 minutes at which point vigorous nitrogen evolution began. Stirring was continued at 60° C. until gas evolution had ceased. 2-(Trimethylsilyl) ethan-1-ol (870 μl, 6.05 mmol) was then added and the mixture was heated at 80° C. for 26 hours. The mixture was allowed to cool to room temperature and concentrated to dryness in vacuo. The residue was loaded onto a 40 g Isco column and eluted with a linear gradient of 0% to 100% EtOAc in hexanes over 18 minutes, detecting with an ELSD. Methyl (1R,5S,6R,7S)-7-(((2-(trimethylsilyl)ethoxy)carbonyl)amino)bicyclo[3.2.0]heptane-6-carboxylate, (400 mg, 1.3 mmol, 84% yield) was isolated as a racemate as a clear, colorless oil. MS (ESI) m′z 314.2 [M+H]⁺. ¹H NMR (500 MHz, CDCl₃) δ 5.68-5.39 (m, 1H), 4.13 (br s, 2H), 4.06-3.94 (m, 1H), 3.69 (s, 3H), 3.00-2.90 (m, 1H), 2.89-2.78 (m, 1H), 2.77-2.63 (m, 1H), 1.78 (br d, J=9.0 Hz, 3H), 1.69-1.40 (m, 3H), 0.97 (br s, 2H), 0.04 (s, 9H).

### Example 1-2-Preparation of 2-(trimethylsilyl)ethyl((1S,5R,6S,7R)-7-((4-fluoro-3-(trifluoromethyl)phenyl)carbamoyl)bicyclo[3.2.0]heptan-6-yl)carbamate

Example 1-1 was dissolved in a mixture of acetonitrile (3.8 mL), water (0.12 mL), and Et₃N (0.53 mL, 3.8 mmol) in a 20 mL vial. LiBr (1.10 g, 12.8 mmol) was added and the reaction mixture was stirred for 18 hours at room temperature. The reaction mixture was then diluted with EtOAc (50 mL) and water (50 mL) and the aqueous layer was then separated and acidified with 1 N HCl and back-extracted with EtOAc. The organic layer was separated, concentrated in vacuo to a tan powder and 2-(trimethylsilyl)ethyl ((1S,5R,6S,7R)-7-((4-fluoro-3-(trifluoromethyl)phenyl)carbamoyl)bicyclo[3.2.0]heptan-6-yl) carbamate was used without further purification in the next step. MS (ESI) m z 300.0 [M+H]⁺. 2-(trimethylsilyl)ethyl ((1S,5R,6S,7R)-7-((4-fluoro-3-(trifluoromethyl)phenyl)carbamoyl)bicyclo[3.2.0]heptan-6-yl) carbamate. (240 mg, 0.80 mmol), 4-fluoro-3-(trifluoromethyl) aniline (144 mg, 0.800 mmol), and Et₃N (0.22 mL, 1.60 mmol) were dissolved in DMF (2 mL). BOP (390 mg, 0.88 mmol) was added and the reaction mixture was stirred for two hours at 65° C. The reaction mixture was allowed to cool to room temperature and diluted with water (25 mL). A precipitate formed which was filtered and dried in vacuo to yield Example 1-2, 2-(trimethylsilyl)ethyl ((1S,5R,6S,7R)-7-((4-fluoro-3-(trifluoromethyl)phenyl)carbamoyl)bicyclo[3.2.0]heptan-6-yl) carbamate (460 mg, 65% yield) was isolated as a tan solid. ¹H NMR (500 MHZ, CDCl₃) δ 7.86-7.78 (m, 1H), 7.75-7.65 (m, 2H), 7.15 (t. J=9.3 Hz, 1H), 5.26-5.15 (m, 1H), 4.13-4.05 (m, 1H), 4.02-3.91 (m, 2H), 3.26-3.17 (m, 1H), 2.97-2.89 (m, 1H), 2.65-2.56 (m, 1H), 1.98-1.87 (m, 1H), 1.83-1.72 (m, 2H), 1.68-1.63 (m, 3H), 1.59-1.49 (m, 1H), 0.87-0.71 (m, 2H), −0.02 (s, 9H).

### Example 1-3-Preparation of (1R,5S,6R,7S)-7-amino-N-(4-fluoro-3-(trifluoromethyl)phenyl)bicyclo[3.2.0]heptane-6-carboxamide, hydrochloride salt

Example 1-2 (57 mg, 0.12 mmol) was dissolved in dioxane (6 mL) and HCl (0.25 ml, 1.0 mmol) was added as a 4M solution in dioxane. The clear solution was stirred for 1 hour then concentrated to dryness under reduced pressure. Racemic Example 1C, (1R,5S,6R,7S)-7-amino-N-(4-fluoro-3-(trifluoromethyl)phenyl)bicyclo[3.2.0]heptane-6-carboxamide hydrochloride (44 mg, 100%) was isolated as a brown solid. MS (ESI) m/z 317.1 [M+H]⁺. ¹H NMR (500 MHz, CD₃OD) δ 8.13 (dd, J=6.4, 2.6 Hz, 1H), 7.86-7.79 (m, 1H), 7.32 (t. J=9.6 Hz, 1H), 3.52 (dd. J=7.8, 4.4 Hz, 1H), 3.11-3.00 (m, 2H), 2.98-2.89 (m, 1H), 2.04-1.93 (m, 1H), 1.92-1.79 (m, 3H), 1.76-1.58 (m, 2H).

### Example 1-4-(1R,5S,6R,7S)-7-(5-bromo-2-methoxybenzamido)-N-(4-fluoro-3-(trifluoromethyl)phenyl)bicyclo[3.2.0]heptane-6-carboxamide

Example 1-3 (100 mg, 0.232 mmol), 5-bromo-2-methoxybenzoic acid (54 mg, 0.23 mmol), Et₃N (0.032 mL, 0.23 mmol), then HATU (88 mg, 0.23 mmol) were dissolved in DMF (2 mL) in a 20 mL vial and the reaction mixture was stirred for 1 hour at room temperature. The reaction mixture was concentrated to dryness under reduced pressure and the residue was purified by prep HPLC with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 44% B, 44-84% B over 20 minutes, then a 10-minute hold at 100% B; Flow Rate: 20 mL/min; Column Temperature: 25 C. Fraction collection was triggered by MS and UV signals. Fractions containing the desired product were combined and dried via centrifugal evaporation. Example 1-1, (1R,5S,6R,7S)-7-(5-Bromo-2-methoxybenzamido)-N-(4-fluoro-3-(trifluoromethyl)phenyl)bicyclo[3.2.0]heptane-6-carboxamide, racemic (118 mg, 96%) was isolated as a white powder. MS (ESI) m/z 529.0 [M+H]⁺. MS (ESI) m/z 531.1 [M+3]⁺. ¹H NMR (500 MHZ, DMSO-d₆) δ 10.26 (s, 1H), 8.88 (br d, J=8.2 Hz, 1H), 8.25-8.13 (m, 1H), 7.79-7.66 (m, 1H), 7.62-7.49 (m, 2H), 7.44 (t, J=9.7 Hz, 1H), 7.06 (d, J=8.8 Hz, 1H), 4.29 (td, J=8.5, 5.1 Hz, 1H), 3.85-3.69 (m, 3H), 3.07 (br dd, J=8.7, 4.1 Hz, 1H), 3.01-2.92 (m, 1H), 2.73 (br d, J=5.1 Hz, 1H), 1.94-1.76 (m, 2H), 1.75-1.62 (m, 2H), 1.60-1.38 (m, 2H).

### Example 1-6-fluoro-3′-(((1S,5R,6S,7R)-7-((4-fluoro-3-trifluoromethyl)phenyl)carbamoyl)bicyclo[3.2.0]heptan-6-yl)carbamoyl)-4′-methoxy-[1,1′-biphenyl]-3-carboxylic acid

Example 1-4 (54 mg, 0.10 mmol) and 3-borono-4-fluorobenzoic acid (19 mg, 0.10 mmol) were dissolved in DMF (2 mL) in a 20 mL vial equipped with a septum sealed cap. 3M aqueous K₃PO₄ (0.10 mL, 0.30 mmol) was added to the solution and the reaction mixture degassed via vacuum and nitrogen back-fill 3 times. XPhos Pd G2 (8 mg, 10.2 μmol) was added under a gentle stream of nitrogen and the reaction mixture was stirred at 75° C. for 30 minutes. The reaction mixture was then concentrated to dryness under reduced pressure and the residue was purified by prep HPLC with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 42% B, 42-82% B over 20 minutes, then a 4-minute hold at 100% B: Flow Rate: 20 mL/min; Column Temperature: 25° C. Fraction collection was triggered by MS signals to yield 6-fluoro-3′-(((1S,5R,6S,7R)-7-((4-fluoro-3-(trifluoromethyl)phenyl)carbamoyl)bicyclo[3.2.0]heptan-6-yl)carbamoyl)-4′-methoxy-[1, l′-biphenyl]-3-carboxylic acid (4.3 mg, 7%) was isolated in 97.1% purity (Method A. Retention Time: 1.78 min). MS (ESI) m/z 589.3 [M+H]⁺. ¹H NMR (500 MHz, DMSO-d₆) δ 10.25 (s, 1H), 8.90 (br d, J=8.3 Hz, 1H), 8.12 (br d, J=3.2 Hz, 1H), 8.02-7.90 (m, 2H), 7.73 (br s, 2H), 7.64 (br d, J=8.6 Hz, 1H), 7.39 (br t, J=9.3 Hz, 1H), 7.34 (br t, J=9.7 Hz, 1H), 7.21 (d, J=8.5 Hz, 1H), 4.39-4.26 (m, 1H), 3.86 (s, 3H), 3.08 (br dd, J=8.9, 4.2 Hz, 1H), 3.02-2.93 (m, 1H), 2.79-2.71 (m, 1H), 1.95-1.76 (m, 2H), 1.73 (br dd, J=12.2, 5.5 Hz, 1H), 1.66 (br dd. J=12.5, 5.7 Hz, 1H), 1.60-1.42 (m, 2H).

### Example 2: (1R,5S,6R,7S)—N-(4-fluoro-3-(trifluoromethyl)phenyl)-7-(5-(3-hydroxyprop-1-yn-1-yl)-2-methoxybenzamido) bicyclo[3.2.0]heptane-6-carboxamide

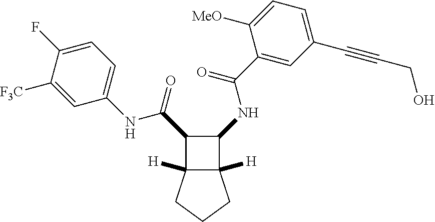 [SMILES: COc1ccc(C#CCO)cc1C(=O)N[C@H]1[C@@H]2CCC[C@@H]2[C@H]1C(=O)Nc1ccc(F)c(C(F)(F)F)c1]

Example 2—Example 2 was prepared using the general procedures described for Example 1-4 by coupling Example 1-3 (100 mg, 0.32 mmol) with 5-(3-hydroxyprop-1-yn-1-yl)-2-methoxybenzoic acid (65 mg, 0.32 mmol). Example 2 (153 mg, 96%) was isolated in 95% purity.

Example 7-Preparation of 2-(6-fluoro-3′-(((1S,5R,6S,7R)-7-((4-fluoro-3-(trifluoromethyl)phenyl)carbamoyl)bicyclo[3.2.0]heptan-6-yl)carbamoyl)-4′-methoxy-[1,1′-biphenyl]-3-yl)-2-((phenylcarbamoyl)oxy) acetic acid

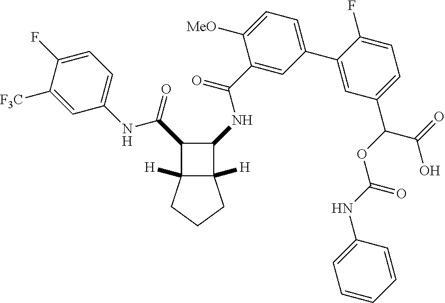 [SMILES: COc1ccc(-c2cc(C(OC(=O)Nc3ccccc3)C(=O)O)ccc2F)cc1C(=O)N[C@H]1[C@@H]2CCC[C@@H]2[C@H]1C(=O)Nc1ccc(F)c(C(F)(F)F)c1]

### Example 7-1-Preparation of tert-butyl 2-(6-fluoro-3′-(((1S,5R,6S,7R)-7-((4-fluoro-3-(trifluoromethyl)phenyl)carbamoyl)bicyclo[3.2.0]heptan-6-yl)carbamoyl)-4′-methoxy-[1, l′-biphenyl]-3-yl)-2-hydroxyacetate

Example 7-1 was prepared using the general coupling procedure described for the preparation of Example 1-4 substituting 5-bromo-2-methoxybenzoic acid for Intermediate 8-2 (43 mg, 0.11 mmol) to yield tert-butyl 2-(6-fluoro-3′-(((1S,5R,6S,7R)-7-((4-fluoro-3-(trifluoromethyl)phenyl)carbamoyl)bicyclo[3.2.0]heptan-6-yl)carbamoyl)-4′-methoxy-[1,1′-biphenyl]-3-yl)-2-hydroxyacetate (61 mg, 80% yield). MS (ESI) m/z 589.3 [M+H]⁺

Example 7-2-tert-butyl 2-(6-fluoro-3′-(((1S,5R,6S,7R)-7-((4-fluoro-3-(trifluoromethyl)phenyl)carbamoyl)bicyclo[3.2.0]heptan-6-yl)carbamoyl)-4′-methoxy-[1,1′-biphenyl]-3-yl)-2-hydroxyacetate (60 mg, 0.089 mmol) and pyridine (0.072 mL, 0.89 mmol) were dissolved in CH₂Cl₂ (4 mL) in a 2 dram vial, followed by the addition of phenyl isocyanate (0.10 mL, 0.89 mmol). The reaction mixture was stirred at room temperature for 19 hours. The reaction mixture was loaded onto a 40 g Isco column and eluted with a linear gradient of 0% to 100% EtOAc in hexanes. MS (ESI) m/z 794.3 (M+H)⁺.

Example 7—Example 7-2 was dissolved in CH₂Cl (4 mL) and TFA (1 mL). The resulting solution was stirred at room temperature for 2 hours. After concentration to dryness under reduced pressure, the residue was purified via preparative LC/MS with the following conditions: Column: XBridge C18, 200 mm×19 mm, 5-μm particles; Mobile Phase A: 5:95 acetonitrile:water with 0.1% trifluoroacetic acid: Mobile Phase B: 95:5 acetonitrile:water with 0.1% trifluoroacetic acid; Gradient: a 0-minute hold at 45% B, 45-85% B over 20 minutes, then a 4-minute hold at 100% B; Flow Rate: 20 mL/min; Column Temperature: 25° C. Fraction collection was triggered by MS and UV signals.

The resulting mixture of diastereomers was were separated by chiral SFC using the following conditions: Column: Chiral OD, 30×250 mm. 5 micron particles; Mobile Phase: 75% CO₂/25% MeOH w/0.1% DEA: Flow Conditions: 100 mL/min Detector Wavelength: 220 nm: Injection Details: 1,500 μL 4.4 mg dissolved in 3 mL MeOH.
Peak 1 eluted at 25.7 minutes
Peak 2 (Example 7) eluted at 31.4 minutes

The peaks were analyzed post-purification for enantiomeric purity using the following analytical conditions: Column: Chiral OD, 4.6×100 mm, 5 micron, Mobile Phase 75% CO₂/25% MeOH w/0.1% DEA, Flow Conditions: 2.0 mL/min, 150 Bar, 40° C., Detector Wavelength: 220 nm, Injection Details: 10 μL of ˜1 mg/mL in MeOH.

Peak 1 (>95% ee) is a diastereomer of Example 7. ¹H NMR (500 MHz, DMSO-d₆) δ 10.30 (s, 1H), 9.87 (br s, 1H), 8.97 (d, J=8.5 Hz, 1H), 8.17 (dd, J=6.6, 2.3 Hz, 1H), 7.89 (s, 1H), 7.80-7.71 (m, 1H), 7.61 (br t, J=9.9 Hz, 2H), 7.53 (br dd, J=5.0, 2.6 Hz, 1H), 7.48 (br d, J=7.9 Hz, 2H), 7.39 (t. J=9.8 Hz, 1H), 7.35-7.26 (m, 3H), 7.23 (d, J=8.9 Hz, IH), 7.01 (t, J=7.3 Hz, 1H), 5.85 (s, 1H), 4.41-4.34 (m, 1H), 3.87 (s, 2H), 3.14-3.07 (m, 1H), 3.03-2.93 (m, 1H), 2.78-2.71 (m, 1H), 1.92-1.79 (m, 2H), 1.75 (br dd, J=12.4, 5.3 Hz, 1H), 1.69 (br dd, J=12.7, 6.0 Hz, 1H), 1.61-1.53 (m, 1H), 1.53-1.45 (m, 1H)

Peak 2 (>95% ee) is Example 7. MS (ESI) m/z 738.1 [M+H]⁺. ¹H NMR (500 MHZ, DMSO-d₆) δ 10.30 (s, 1H), 9.94-9.78 (m, 1H), 8.96 (d, J=8.5 Hz, 1H), 8.17 (dd, J=6.4, 2.4 Hz, 1H), 7.88 (s, 1H), 7.79-7.71 (m, 1H), 7.66-7.57 (m, 2H), 7.56-7.51 (m, 1H), 7.48 (br d, J=7.6 Hz, 2H), 7.42-7.35 (m, 1H), 7.34-7.26 (m, 3H), 7.26-7.20 (m, 1H), 7.06-6.97 (m, 1H), 5.85 (s, 1H), 4.41-4.32 (m, 1H), 3.87 (s, 2H), 3.14-3.06 (m, 1H), 3.01-2.92 (m, 1H), 2.79-2.70 (m, 1H), 1.93-1.78 (m, 2H), 1.79-1.71 (m, 1H), 1.72-1.64 (m, 1H), 1.62-1.53 (m, 1H), 1.53-1.44 (m, 1H), 1.28-1.20 (m, 1H).

Examples 2, 3, and 5 in Table 1 below were prepared by the general procedures described for Examples 1 & 7.

| | | | MS | | LC RT |
|---|---|---|---|---|---|
| Ex. | | | (ESI) | | Meth. |
| No. | Structure | Name | (M + H) | ¹H NMR | (min) |
| 2 | 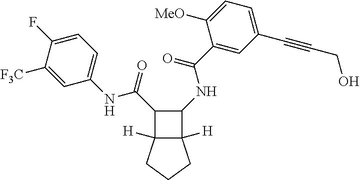 [SMILES: COc1ccc(C#CCO)cc1C(=O)NC1C2CCCC2C1C(=O)Nc1ccc(F)c(C(F)(F)F)c1] | (1R,5S,6R,7S)-N- (4-fluoro-3-(tri- fluoromethyl)- phenyl)-7-(5-(3- hydroxyprop-1- yn-1-yl)-2-meth- oxybenzamido)- bicyclo[3.2.0] heptane-6-carbox- amide, racemic | 505.3 | ¹H NMR (500 MHz, CD₃CN) δ 8.66 (br d, J = 7.6 Hz, 1H), 8.43 (br s, 1H), 8.00 (dd, J = 6.4, 2.7 Hz, 1H), 7.94 (d, J = 2.3 Hz, 1H), 7.72-7.64 (m, 1H), 7.49 (dd, J = 8.7, 2.3 Hz, 1H), 7.22 (t, J = 9.6 Hz, 1H), 7.02 (d, J = 8.7 | 9.62, C |
| | | | | Hz, 1H), 4.43 (td, J = | |
| | | | | 8.1, 5.0 Hz, 1H), 4.35 | |
| | | | | (s, 2H), 3.89 (s, 3H), | |
| | | | | 3.12-3.02 (m, 2H), | |
| | | | | 2.80-2.72 (m, 1H), | |
| | | | | 1.85-1.77 (m, 1H), | |
| | | | | 1.77-1.70 (m, 1H), | |
| | | | | 1.69-1.50 (m, 3H) | |
| 3 | 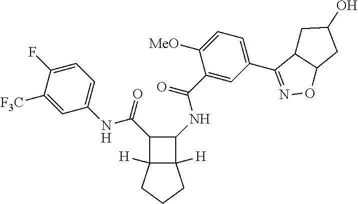 [SMILES: COc1ccc(C2=NOC3CC(O)CC23)cc1C(=O)NC1C2CCCC2C1C(=O)Nc1ccc(F)c(C(F)(F)F)c1] | (1R,5S,6R,7S)-N- (4-fluoro-3-(tri- fluoromethyl)- phenyl)-7-(5-(5- hydroxy-3a,5,6,6a- tetrahydro-4H- cyclopenta[d]isox- azol-3-yl)-2-meth- oxybenzamido)- bicyclo[3.2.0] heptane-6-carbox- amide | 576.3 | ¹HNMR (500 MHz, DMSO-d₆) δ 10.28 (s, 1H), 8.92 (br d, J = 8.4 Hz, 1H), 8.14 (br d, J = 2.2 Hz, 1H), 8.02-7.93 (m, 1H), 7.80-7.65 (m, 2H), 7.42 (br t, J = 9.6 Hz, 1H), 7.17 (d, J = 8.8 Hz, 1H), 5.11 (br t, J = 6.6 Hz, 1H), 4.33 (br d, J = 5.1 Hz, 1H), 4.21-4.02 (m, | 1.95, B |
| | | | | 2H), 3.84 (d, J = 4.1 | |
| | | | | Hz, 3H), 3.08 (dd, | |
| | | | | J = 8.9, 4.0 Hz, 1H), | |
| | | | | 2.95 (br d, J = 4.0 | |
| | | | | Hz, 1H), 2.72 (br d, | |
| | | | | J = 5.4 Hz, 1H), 2.55 | |
| | | | | (s, 3H), 2.02-1.93 | |
| | | | | (m, 1H), 1.87 (br dd, | |
| | | | | J = 13.0, 6.6 Hz, 4H), | |
| | | | | 1.76-1.62 (m, 3H), | |
| | | | | 1.61-1.40 (m, 2H) | |
| 5 | 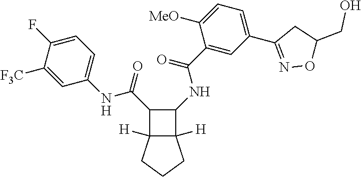 [SMILES: COc1ccc(C2=NOC(CO)C2)cc1C(=O)NC1C2CCCC2C1C(=O)Nc1ccc(F)c(C(F)(F)F)c1] | (1R,5S,6R,7S)-N- (4-fluoro-3-(tri- fluoromethyl)- phenyl)-7-(5-(5- (hydroxymethyl)- 4,5-dihydroisox- azol-3-yl)-2-meth- oxybenzamido)- bicyclo[3.2.0] heptane-6-carbox- amide | 550.3 | ¹HNMR (500 MHz, DMSO-d₆) δ 10.28 (s, 1H), 8.92 (br d, J = 8.0 Hz, 1H), 8.13 (br s, 1H), 7.96 (s, 1H), 7.80-7.68 (m, 2H), 7.42 (br t, J = 9.6 Hz, 1H), 7.16 (d, J = 8.6 Hz, 1H), 4.67 (br dd, J = 11.1, 7.0 Hz, 1H), 4.33 (br d, | 1.96, B |
| | | | | J = 5.2 Hz, 1H), 3.84 | |
| | | | | (s, 2H), 3.35-3.24 (m, | |
| | | | | 1H), 3.13-3.04 (m, | |
| | | | | 2H), 2.99-2.91 (m, | |
| | | | | 1H), 2.72 (br d, J = | |
| | | | | 5.7 Hz, 1H), 2.51 (s, | |
| | | | | 3H), 1.86 (br s, 2H), | |
| | | | | 1.76-1.63 (m, 2H), | |
| | | | | 1.47 (br s, 2H) | |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A compound of Formula (I): 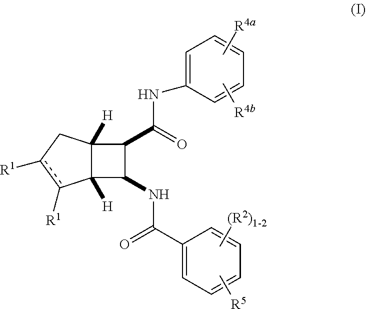
or a pharmaceutically acceptable salt thereof, wherein: [symbol]
is an optional bond;
R¹ is H or halo; or R¹ and R¹ together form a phenyl ring;
R² is halo, C₁₋₄ alky, OH, or —OC₁₋₄ alkyl substituted with 0-4 halo, OH, or —OC₁₋₄ alkyl;
R^{4a} is halo;
R^{4b} is C₁₋₄ alkyl substituted with 0-4 halo;
R⁵ is C₂₋₈ alkenyl substituted with 0-3 R⁶ and 0-2 R⁷, C₂₋₈ alkynyl substituted with 0-3 R⁶ and 0-2 R⁷, C₆₋₁₂ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷; wherein said heterocyclyl is bonded to the phenyl moiety through a carbon or nitrogen atom;
R⁶ is halo, —O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{c};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl;
R^{g} is halo, CN, OH, C₁₋₆ alkyl, C₃₋₆ cycloalkyl, or aryl;
n is zero, 1, 2, or 3; and
p is zero, 1, or 2.

2. The compound of claim 1, having Formula (II): 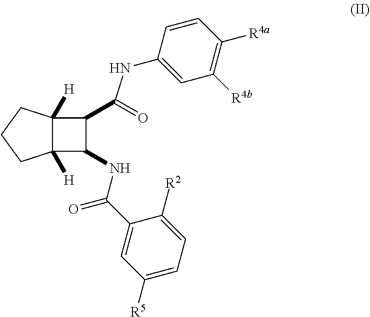
or a pharmaceutically acceptable salt thereof, wherein:
R² is —OC₁₋₄ alkyl substituted with 0-4 halo;
R^{4a} is halo;
R^{4b} is C₁₋₃ alkyl substituted with 0-4 F;
R⁵ is C₂₋₆ alkynyl substituted with 0-3 R⁶ and 0-2 R⁷, C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, CN, C₁₋₃ alkyl, —OH, or —OC₁₋₄ alkyl;
R⁷ is C₁₋₂ alkyl substituted with 0-1 R⁸ and 0-1 R⁹, ORE, —NR^{a}R^{a}, —NR^{a}C(═O)R^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, or —S(═O)ₚNR^{a}R^{a};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{e}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, or —C(═O)NR^{a}R^{a};
R^{a} is H, C₁₋₅ alkyl substituted with 0-4 R^{e}, C₂₋₅ alkenyl substituted with 0-4 R^{e}, C₂₋₃ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₂₋₅ alkenyl substituted with 0-4 R^{e}, C₂₋₅ alkynyl substituted with 0-4 R^{e}, C₃₋₆ carbocyclyl, or heterocyclyl;
R^{d} is H or C₁₋₂ alkyl;
R^{e} is halo, CN, ═O, C₁₋₆ alkyl substituted with 0-5 R⁸, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl;
R^{g} is halo, CN, OH, C₁₋₆ alkyl, or C₃₋₆ cycloalkyl;
n is zero, 1, 2, or 3; and
p is zero, 1, or 2.

3. The compound of claim 2, having Formula (III): 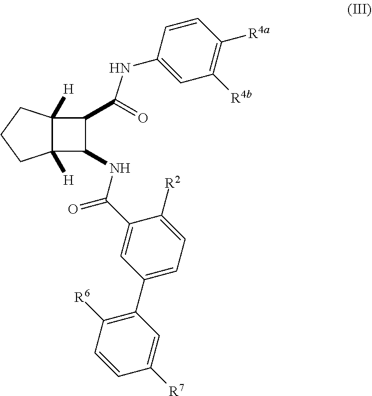
or a pharmaceutically acceptable salt thereof, wherein:
R² is —OC₁₋₃ alkyl;
R^{4a} is F;
R⁶ is CF₃;
R⁶ is halo;
R⁷ is C₁₋₂ alkyl substituted with 0-1 R⁸ and 0-1 R⁹, —C(═O)OR^{b}, or —C(═O)NR^{a}R^{a};
R⁸ is —C(═O)OR^{b}, —C(═O)NHR^{a}, or C₁₋₄ alkyl substituted with 0-3 halo or OH;
R⁹ is —OR^{b}, —NR^{a}R^{a}, —NR^{a}C(═O)R^{b}, or —OC(═O)NR^{a}R^{a};
R^{a} is H, C₁₋₄ alkyl substituted with 0-3 R^{e}, —(CH₂)ₙ—C₃₋₆ cycloalkyl substituted with 0-3 R^{e}, or phenyl substituted with 0-3 R^{e};
R^{b} is H or heterocyclyl substituted with 0-3 R^{e};
R^{e} is halo, CN, ═O, or C₁₋₄ alkyl; and
n is zero or 1.

4. The compound of claim 2, or a pharmaceutically acceptable salt thereof, wherein:
R² is —OCH₃;
R^{4a} is F;
R^{4b} is CF₃;
R⁵ is 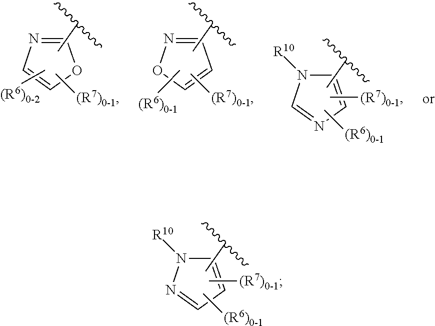
R⁶ is halo, —OH, or C₁₋₄ alkyl substituted with 0-1 OH;
R⁷ is C₁₋₂ alkyl substituted with 0-1 R⁸ and 0-1 R⁹;
R⁸ is —C(═O)OR^{b}, —C(═O)NHR^{a}, or —C(═O)NHOR^{b};
R⁹ is —ORD or —NR^{a}R^{a};
R¹⁰ is H, —C(═O)R^{b}, or C₁₋₄ alkyl substituted with 0-1 R¹;
R¹¹ is —OH, —C(═O)OH, or aryl;
R^{a} is H or C₁₋₃ alkyl; and
R^{b} is H or C₁₋₃ alkyl.

5. The compound of claim 2, or a pharmaceutically acceptable salt thereof, wherein:
R² is —OCH₃;
R^{4a} is F;
R^{4b} is CF₃;
R⁵ is 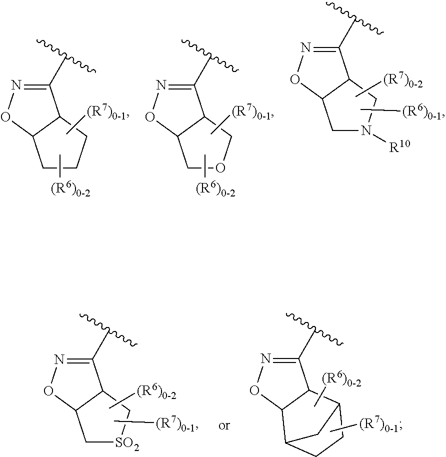
R⁶ is halo, C₁₋₄ alkyl, —OH, or —OC₁₋₄ alkyl;
R⁷ is C₁₋₄ alkyl substituted with 0-1 R⁸ and 0-1 R⁹;
R⁸ is —C(═O)OR^{b};
R⁹ is OH;
R¹⁰ is H, C₁₋₃ alkyl substituted with 0-2 R¹¹, or —C(═O) OC₁₋₄ alkyl;
R¹¹ is —OH, —C(═O)OH, or aryl; and
R^{b} is H or C₁₋₃ alkyl.

6. The compound of claim 2, or a pharmaceutically acceptable salt thereof, wherein:
R² is —OCH₃;
R^{4a} is F;
R^{4b} is CF₃;
R⁵ is C₂₋₅ alkynyl substituted with 0-1 R⁷;
R⁷ is —OR^{b};
R^{b} is H, C₁₋₃ alkyl, or phenyl substituted with 0-2 R^{e};
R^{e} is halo, C₁₋₃ alkyl, or C(═O)OR^{f}; and
R^{f} is H or C₁₋₃ alkyl.

7. A pharmaceutical composition comprising the compound of claim 1 and a pharmaceutically acceptable carrier.

8. A method for treating a disease associated with relaxin comprising administering a therapeutically effective amount of the composition of claim 7 to a patient in need thereof.

9. The method of claim 8 wherein the disease is selected from the group consisting of angina pectoris, unstable angina, myocardial infarction, heart failure, acute coronary disease, acute heart failure, chronic heart failure, and cardiac iatrogenic damage.

10. The method of claim 9 wherein the disease is heart failure.

11. The method of claim 8 wherein the disease is fibrosis.
